(19) United States
(12) Patent Application Publication
(10) Pub. No.: US 2019/0293653 A1
(43) Pub. Date: Sep. 26, 2019

(54) URINE METABOLITE MARKER FOR CHILDHOOD CANCER

(71) Applicants: HITACHI, LTD., Tokyo (JP); National University Corporation Nagoya University, Aichi (JP)

(72) Inventors: Hiroo Uchida, Aichi (JP); Akinari Hinoki, Aichi (JP); Minoru Sakairi, Tokyo (JP); Mayumi Abe, Tokyo (JP)

(73) Assignees: HITACHI, LTD., Tokyo (JP); National University Corporation Nagoya University, Aichi (JP)

(21) Appl. No.: 16/296,515

(22) Filed: Mar. 8, 2019

(30) Foreign Application Priority Data
Mar. 23, 2018 (JP) 2018-055998

(51) Int. Cl. G01N 33/574; G01N 33/68

(52) U.S. Cl. CPC G01N 33/57484; G01N 33/6848

(57) ABSTRACT

Provided is a method, a device and a kit for detecting childhood cancer, predicting the risk of childhood cancer, determining the stage of the childhood cancer, determining the prognosis of the childhood cancer, and/or monitoring the effect of a treatment for the childhood cancer in a subject by measuring ae urinary metabolite of the subject.

### RELATED APPLICATION

This application claims the benefit of priority to Japanese Patent Application number 2018-055998, filed Mar. 23, 2018, hereby incorporated by reference in its entirety.

### TECHNICAL FIELD

The present invention relates to a method, a kit and a device for evaluating a disease of a subject based on metabolite information in the urine of a subject, specifically, the condition of a childhood cancer.

### BACKGROUND ART

“Childhood cancers” are the number one cause of death in children, but measures for treating these cancers are still insufficient. Generally, most “childhood cancers” have better treatment results if the disease is in an early stage, and it has been verified that early detection is directly linked to the prognosis. However, early diagnosis is very difficult, and often results in infiltration into other organs and distant metastasis by the time of detection, thus, there are numerous cases in which treatment becomes difficult. Further, when surgical resection is difficult or when there is the possibility of residual tumors even with surgical resection, chemotherapy and radiation therapy are often performed. In tumors in which no useful tumor marker has been found, there are no methods for making an effective determination other than by diagnostic imaging. Such tumors include, for example, neuroblastoma, Wilms tumor, rhabdomyosarcoma, osteosarcoma, and the Ewing sarcoma family of tumors. For the evaluation of residual tumors, image examination such as examinations by Computed Tomography (CT) and Positron Emission Tomography (PET) are effective, but in cases when an evaluation cannot be made even with these examinations, a biopsy or resection of the residual mass must be performed, and as a result, a largely invasive procedure is required. On the other hand, even though residual tumors can be evaluated with PET examination and the like, many “childhood cancers” require image diagnosis be continued over a long period of time, and there is debate with regards to the carincogenicity of these examinations themselves.

For example, methods and devices for detecting catecholamine metabolites (vanillylmandelate (VMA) and homovanillate (HVA)) as the urinary metabolite for the examination of cancers such as neuroblastoma which is one kind of childhood cancer have been reported (for example, Japanese Unexamined Patent Application Publication Hei 5-113438; Japanese Examined Patent Application Publication Hei 7-92456; and Japanese Examined Patent Application Publication Hei 6-58364). These biomarkers may be useful, but there are cases in which the cancers are not detected.

### SUMMARY

As urinary metabolites are structurally stable against the influence of enzymes compared to substances in the blood, there is sufficient likelihood that the urinary metabolites can be used as tumor markers. In addition, urinary markers are easy to collect even from children, as urine is used as the specimen, and are remarkably useful in a screening application for cancer. Therefore, the object of the present invention is to identify new urinary markers for childhood cancers, and use the new urinary markers in the evaluation of the childhood cancers such as in a childhood cancer examination.

The present inventors comprehensively analyzed the urinary metabolites of children having a childhood cancer and the urinary metabolites of healthy children of the same age by a liquid chromatography mass spectrometer (LC/MS: Liquid Chromatograph/Mass Spectrometer), searched for tumor markers, identified a plurality of metabolites of which levels are different between the healthy children and the childhood cancer patients, and based on a multivariate analysis, succeeded in identifying promising urinary tumor markers for the evaluation of the childhood cancer.

Namely, the present invention relates to a method, a device and a kit for detecting childhood cancer (specifically, neuroblastoma), predicting the risk of the childhood cancer, determining the stage of the childhood cancer, determining the prognosis of the childhood cancer, and/or monitoring the results of a treatment for the childhood cancer in a subject by measuring the urinary metabolite(s) of the subject.

In an embodiment, the present disclosure provides a method for evaluating a childhood cancer, including:

a step of measuring a urinary metabolite in a urine sample derived from a subject, and

a step of evaluating a childhood cancer in the subject on the basis of the result of the measurement,

wherein the urinary metabolite includes at least one metabolite selected from the group consisting of:

(i) at least one metabolite selected from the group consisting of 3-methoxytyramine sulfate, vanillactate, homovanillate, vanillylmandelate, 3,4-dihydroxyphenylacetate, phenol glucuronide, 3,4-dihydroxyphenylacetate sulfate, 3-methoxytyrosine, 3-methoxytyramine, 3-methoxy-4-hydroxyphenylglycol, and dopamine;

(ii) at least one metabolite selected from the group consisting of N-acetylcysteine, cystathionine, and S-adenosylhomocysteine;

(iii) at least one metabolite selected from the group consisting of cortisol 21-glucuronide and cortisol; and

(iv) a metabolite which is xanthopterin,

provided that if the urinary metabolite to be evaluated is only one, it is not homovanillate or vanillylmandelate.

In another embodiment, the present disclosure provides a device for evaluating a childhood cancer, including:

a measurement unit which is configured to measure a urinary metabolite in a urine sample,

a comparison unit which is configured to compare a measurement value of the urinary metabolite measured by the measurement unit with a reference value or a previous measurement value, and

a determination unit which is configured to evaluate the childhood cancer from a comparison result obtained by the comparison unit,

wherein the urinary metabolite includes at least one metabolite selected from the group consisting of:

(i) at least one metabolite selected from the group consisting of 3-methoxytyramine sulfate, vanillactate, homovanillate, vanillylmandelate, 3,4-dihydroxyphenylacetate, phenol glucuronide, 3,4-dihydroxyphenylacetate sulfate, 3-methoxytyrosine, 3-methoxytyramine, 3-methoxy-4-hydroxyphenylglycol, and dopamine;

(ii) at least one metabolite selected from the group consisting of N-acetylcysteine, cystathionine, and S-adenosylhomocysteine;

(iii) at least one metabolite selected from the group consisting of cortisol 21-glucuronide and cortisol; and

(iv) a metabolite which is xanthopterin,

provided that if the urinary metabolite to be evaluated is only one, it is not homovanillate or vanillylmandelate.

Further, in another embodiment, the disclosure provides a method for evaluating the efficacy of a treatment for a childhood cancer, including:

a step of measuring a urinary metabolite in a urine sample from an animal having a childhood cancer treated with a therapeutic drug or a therapeutic method to be tested, and

a step of evaluating, on the basis of the result of the measurement, the efficacy of the therapeutic drug or the therapeutic method to be tested for the childhood cancer,

wherein the urinary metabolite includes at least one metabolite selected from the group consisting of:

(i) at least one metabolite selected from the group consisting of 3-methoxytyramine sulfate, vanillactate, homovanillate, vanillylmandelate, 3,4-dihydroxyphenylacetate, phenol glucuronide, 3,4-dihydroxyphenylacetate sulfate, 3-methoxytyrosine, 3-methoxytyramine, 3-methoxy-4-hydroxyphenylglycol, and dopamine;

(ii) at least one metabolite selected from the group consisting of N-acetylcysteine, cystathionine, and S-adenosylhomocysteine;

(iii) at least one metabolite selected from the group consisting of cortisol 21-glucuronide and cortisol; and

(iv) a metabolite which is xanthopterin,

provided that if the urinary metabolite to be evaluated is only one, it is not homovanillate or vanillylmandelate.

The present invention provides a method, a device and a kit for evaluating a childhood cancer which is minimally invasive, simple, and inexpensive. Since urine is examined, the collection method at a clinical site is also very simple, and the convenience for the medical staff also greatly improves. Therefore, the present invention is advantageous in fields such as the detection of childhood cancers, childhood cancer examination, treatment evaluation, and drug discovery.

### BRIEF DESCRIPTION OF THE DRAWINGS

FIG. 1 is an analysis flow example for extracting urinary tumor marker candidates.

FIG. 2 is an analysis flow example by a cancer examination model.

FIG. 3 is a graph illustrating the predicted value when a urinary tumor marker (3-methoxytyramine sulfate) is used in a cancer examination model.

FIG. 4 is a graph illustrating the predicted value when a urinary tumor marker (vanillactate) is used in the cancer examination model.

FIG. 5 is a graph illustrating the predicted value when a urinary tumor marker (homovanillate) is used in the cancer examination model.

FIG. 6 is a graph illustrating the predicted value when a urinary tumor marker (vanillylmandelate) is used in the cancer examination model.

FIG. 7 is a graph illustrating the predicted value when a urinary tumor marker (3,4-dihydroxyphenylacetate) is used in the cancer examination model.

FIG. 8 is a graph illustrating the predicted value when a urinary tumor marker (phenol glucuronide) is used in the cancer examination model.

FIG. 9 is a graph illustrating the predicted value when a urinary tumor marker (3,4-dihydroxyphenylacetate sulfate) is used in the cancer examination model.

FIG. 10 is a graph illustrating the predicted value when a urinary tumor marker (3-methoxytyrosine) is used in the cancer examination model.

FIG. 11 is a graph illustrating the predicted value when a urinary tumor marker (3-methoxytyramine) is used in the cancer examination model.

FIG. 12 is a graph illustrating the predicted value when a urinary tumor marker (3-methoxy-4-hydroxyphenylglycol) is used in the cancer examination model.

FIG. 13 is a graph illustrating the predicted value when a urinary tumor marker (dopamine) is used in the cancer examination model.

FIG. 14 is a graph illustrating the predicted value when a urinary tumor marker

(N-acetylcysteine) is used in the cancer examination model.

FIG. 15 is a graph illustrating the predicted value when a urinary tumor marker (cystathionine) is used in the cancer examination model.

FIG. 16 is a graph illustrating the predicted value when a urinary tumor marker

(S-adenosylhomocysteine) is used in the cancer examination model.

FIG. 17 is a graph illustrating the predicted value when a urinary tumor marker (cortisol 21-glucuronide) is used in the cancer examination model.

FIG. 18 is a graph illustrating the predicted value when a urinary tumor marker (cortisol) is used in the cancer examination model.

FIG. 19 is a graph illustrating the predicted value when a urinary tumor marker (xanthopterin) is used in the cancer examination model.

FIG. 20 is a graph illustrating the predicted value when the urinary tumor markers (3-methoxytyramine sulfate and vanillactate) are used in the cancer examination model.

FIG. 21 is a graph illustrating the predicted value when the urinary tumor markers (3-methoxytyramine sulfate and homovanillate) are used in the cancer examination model.

FIG. 22 is a graph illustrating the predicted value when the urinary tumor markers (3-methoxytyramine sulfate and vanillylmandelate) are used in the cancer examination model.

FIG. 23 is a graph illustrating the predicted value when the urinary tumor markers (3-methoxytyramine sulfate and 3,4-dihydroxyphenylacetate) are used in the cancer examination model.

FIG. 24 is a graph illustrating the predicted value when the urinary tumor markers (3-methoxytyramine sulfate and phenol glucuronide) are used in the cancer examination model.

FIG. 25 is a graph illustrating the predicted value when the urinary tumor markers (3-methoxytyramine sulfate and 3-methoxytyrosine) are used in the cancer examination model.

FIG. 26 is a graph illustrating the predicted value when the urinary tumor markers (3-methoxytyramine sulfate and dopamine) are used in the cancer examination model.

FIG. 27 is a graph illustrating the predicted value when the urinary tumor markers (homovanillate and vanillylmandelate) are used in the cancer examination model.

FIG. 28 is a graph illustrating the predicted value when the urinary tumor markers (homovanillate and 3-methoxytyrosine) are used in the cancer examination model.

FIG. 29 is a graph illustrating the predicted value when the urinary tumor markers (vanillylmandelate and 3-methoxytyramine) are used in the cancer examination model.

FIG. 30 is a graph illustrating the predicted value when the urinary tumor markers (homovanillate, vanillylmandelate and 3-methoxytyramine sulfate) are used in the cancer examination model.

FIG. 31 is a graph illustrating the predicted value when the urinary tumor markers

(N-acetylcysteine and cystathionine) are used in the cancer examination model.

FIG. 32 is a graph illustrating the predicted value when the urinary tumor markers (cystathionine, and S-adenosylhomocysteine) are used in the cancer examination model.

FIG. 33 is a graph illustrating the predicted value when the urinary tumor markers (cortisol 21-glucuronide and cortisol) are used in the cancer examination model.

FIG. 34 is a graph illustrating the predicted value when the urinary tumor markers (3-methoxytyramine sulfate and N-acetylcysteine) are used in the cancer examination model.

FIG. 35 is a graph illustrating the predicted value when the urinary tumor markers (3-methoxytyramine sulfate and cystathionine) are used in the cancer examination model.

FIG. 36 is a graph illustrating the predicted value when the urinary tumor markers (3-methoxytyramine sulfate and S-adenosylhomocysteine) are used in the cancer examination model.

FIG. 37 is a graph illustrating the predicted value when the urinary tumor markers (homovanillate and N-acetylcysteine) are used in the cancer examination model.

FIG. 38 is a graph illustrating the predicted value when the urinary tumor markers (homovanillate and cystathionine) are used in the cancer examination model.

FIG. 39 is a graph illustrating the predicted value when the urinary tumor markers (homovanillate, vanillylmandelate and cystathionine) are used in the cancer examination model.

FIG. 40 is a graph illustrating the predicted value when the urinary tumor markers (3-methoxytyramine sulfate and cortisol 21-glucuronide) are used in the cancer examination model.

FIG. 41 is a graph illustrating the predicted value when the urinary tumor markers (3-methoxytyramine sulfate and cortisol) are used in the cancer examination model.

FIG. 42 is a graph illustrating the predicted value when the urinary tumor markers (homovanillate, vanillylmandelate and cortisol 21-glucuronide) are used in the cancer examination model.

FIG. 43 is a graph illustrating the predicted value when the urinary tumor markers (homovanillate, vanillylmandelate, 3-methoxytyramine sulfate and cortisol 21-glucuronide) are used in the cancer examination model.

FIG. 44 is a graph illustrating the predicted value when the urinary tumor markers (homovanillate, vanillylmandelate, 3-methoxytyramine sulfate, cystathionine, N-acetylcysteine and cortisol 21-glucuronide) are used in the cancer examination model.

FIG. 45 is a graph illustrating the predicted value when the urinary tumor markers (homovanillate, vanillylmandelate, 3-methoxytyramine sulfate and cortisol) are used in the cancer examination model.

FIG. 46 is a graph illustrating the predicted value when the urinary tumor markers (homovanillate, vanillylmandelate, 3-methoxytyramine sulfate, cystathionine, N-acetylcysteine and cortisol 21-glucuronide) are used in the cancer examination model by using a urine specimen from a child with a childhood cancer prior to treatment and after treatment.

FIG. 47 illustrates a constitution example of the device according to the present invention.

### DETAILED DESCRIPTION

New urinary tumor markers and marker groups related to childhood cancers (specifically, neuroblastoma) are utilized in the method, device and kit according to the present invention. The urinary tumor marker(s) is(are) a metabolite(s) whose level in urine changes with the development and progression of childhood cancer and before and after treatment, and thus, is(are) useful for the detection of childhood cancer, the prediction of the risk of childhood cancer, the determination of the stage of the childhood cancer, the determination of the prognosis of the childhood cancer, and/or the monitoring of the effect of treatment for childhood cancer.

The urinary metabolites used as the markers in the present invention are structurally stable against the influence of enzymes compared to substances in the blood, thus, the convenience as tumor markers may be high. In addition, as urine is used as the specimen, it can easily be collected even from children, and urine is remarkably useful in a screening application for cancer.

The method for evaluating a childhood cancer according to the present invention includes a step of measuring a urinary metabolite(s) in a urine sample derived from a subject, and evaluating the childhood cancer in the subject on the basis of the result of the measurement.

The terms “urinary metabolite” or “urinary tumor marker” or “biomarker” mean the urinary metabolites to be measured in order to detect a childhood cancer, i.e., the urinary metabolite(s) listed in the following table. Further, the term “marker group” is a combination including two or more the urinary tumor markers. The expression “measuring” means obtaining the relative abundance or the absolute concentration of the urinary metabolite in the urine sample. The relative abundance is the ratio of the measurement strength of the target metabolite(s) relative to a reference substance which is intentionally added. On the other hand, the absolute concentration is obtained by a method including creating a calibration curve (the relationship between the concentration of the metabolite and the measurement strength of the metabolite) using the same metabolite in advance for the target metabolite, and calculating the absolute concentration thereof from the measured strength. Further, in the present invention, the expression “measure the urinary tumor marker” means that the metabolite which is a urinary tumor marker may be measured, or the secondary substance or the derivative thereof may be measured. The “secondary substance” and the “derivative” respectively mean a substance secondarily produced from the metabolite which is a urinary tumor marker and a substance derived from the metabolite. Examples of the “secondary substance” and the “derivative” include, but are not limited to fragments of the metabolite, modified metabolites and the like.

The main urinary tumor markers used in the present invention are summarized in the following Table 1. The urinary metabolites from 58 healthy children and 6 childhood cancer patients (neuroblastoma) are exhaustively analyzed, and 30 important metabolites are extracted by Wilcoxon rank sum test and Random Forest analysis for the results. Thereamong, only the metabolites extracted having known structures and known metabolic pathways are shown in Table 1. In the table, as the result of retrieval from a database, the name of the metabolites whose structures are known are shown in the “Metabolite” column. Further, the “Mass” column shows the mass when detected by the detection method described in the “Detection platform” column. “LC/MS Neg” and “LC/MS Pos” in the “Detection platform” column respectively refer to the “Negative ion detection mode of the liquid chromatography mass spectrometer (LC/MS)” and the “Positive ion detection mode of the liquid chromatography mass spectrometer (LC/MS)”. Note that, the table describes one of LC/MS positive ion detection mode or LC/MS negative ion detection mode as the detection platform, but depending on the device to be used, there are cases when the positive ion detection mode and the negative ion detection mode can be reversed at a high speed, and in this case, both of the positive ion detection mode and the negative ion detection mode are described as the detection platform.

TABLE 1
Urinary tumor markers

| | | | Detection |
|---|---|---|---|
| Metabolite | Metabolic pathway | Mass | platform |
| N-acetylcysteine | Methionine | 162.02304 | LC/MS Neg |
| | metabolism | | |
| 3-methoxytyramine | Tyrosine metabolism | 246.04416 | LC/MS Neg |
| sulfate | | | |
| vanillactate | Tyrosine metabolism | 211.06119 | LC/MS Neg |
| xanthopterin | Pterin metabolism | 180.0516 | LC/MS Pos |
| Homovanillate (HVA) | Tyrosine metabolism | 181.05063 | LC/MS Neg |
| Vanillylmandelate | Tyrosine metabolism | 197.04555 | LC/MS Neg |
| (VMA) | | | |
| 3,4- | Tyrosine metabolism | 123.04515 | LC/MS Neg |
| dihydroxyphenylacetate | | | |
| cystathionine | Methionine | 223.07471 | LC/MS Pos |
| | metabolism | | |
| phenol glucuronide | Tyrosine metabolism | 269.06667 | LC/MS Neg |
| cortisol 21-glucuronide | Corticosteroid | 537.23413 | LC/MS Neg |
| cortisol | Corticosteroid | 361.20204 | LC/MS Neg |
| S-adenosylhomocysteine | Methionine | 383.11431 | LC/MS Neg |
| (SAH) | metabolism | | |
| 3,4- | Tyrosine metabolism | 246.99179 | LC/MS Neg |
| dihydroxyphenylacetate | | | |
| sulfate | | | |
| 3-methoxytyrosine | Tyrosine metabolism | 212.09174 | LC/MS Pos |
| 3-methoxytyramine | Tyrosine metabolism | 168.10191 | LC/MS Pos |
| 3-methoxy-4- | Tyrosine metabolism | 167.07028 | LC/MS Pos |
| hydroxyphenylglycol | | | |
| dopamine | Tyrosine metabolism | 154.08626 | LC/MS Pos |

Further, the metabolites shown in Table 1 can be divided into the metabolites related to tyrosine metabolism, the metabolites related to methionine, cysteine, and SAM metabolism (in the description, abbreviated as “methionine metabolism”), the metabolites related to corticosteroid, and the metabolite related to pterin metabolism as shown in the item “Metabolic pathway” of Table 1.

In one embodiment, 3-methoxytyramine sulfate shown in Table 1 may be measured as the urinary tumor marker. Namely, the compound whose mass is measured as 246.04416 is measured in the LC/MS negative ion detection mode.

In one embodiment, cortisol shown in Table 1 may be measured as the urinary tumor marker. Namely, the compound whose mass is measured as 361.20204 is measured in the LC/MS negative ion detection mode.

In one embodiment, cortisol 21-glucuronide shown in Table 1 may be measured as the urinary tumor marker. Namely, the compound whose mass is measured as 537.23413 is measured in the LC/MS negative ion detection mode.

In one embodiment, N-acetylcysteine shown in Table 1 may be measured as the urinary tumor marker. Namely, the compound whose mass is measured as 162.02304 is measured in the LC/MS negative ion detection mode.

In one embodiment, 3-methoxytyrosine shown in Table 1 may be measured as the urinary tumor marker. Namely, the compound whose mass is measured as 212.09174 is measured in the LC/MS positive ion detection mode.

In one embodiment, phenol glucuronide shown in Table 1 may be measured as the urinary tumor marker. Namely, the compound whose mass is measured as 269.06667 is measured in the LC/MS negative ion detection mode.

In one embodiment, 3,4-dihydroxyphenylacetate shown in Table 1 may be measured as the urinary tumor marker. Namely, the compound whose mass is measured as 123.04515 is measured in the LC/MS negative ion detection mode.

In one embodiment, 3,4-dihydroxyphenylacetate sulfate shown in Table 1 may be measured as the urinary tumor marker. Namely, the compound whose mass is measured as 246.99179 is measured in the LC/MS negative ion detection mode.

In one embodiment, homovanillate shown in Table 1 may be measured as the urinary tumor marker. Namely, the compound whose mass is measured as 181.05063 is measured in the LC/MS negative ion detection mode.

In one embodiment, xanthopterin shown in Table 1 may be measured as the urinary tumor marker. Namely, the compound whose mass is measured as 180.0516 is measured in the LC/MS positive ion detection mode.

In one embodiment, vanillactate shown in Table 1 may be measured as the urinary tumor marker. Namely, the compound whose mass is measured as 211.06119 is measured in the LC/MS negative ion detection mode.

In one embodiment, 3-methoxytyramine shown in Table 1 may be measured as the urinary tumor marker. Namely, the compound whose mass is measured as 168.10191 is measured in the LC/MS positive ion detection mode.

In one embodiment, vanillylmandelate shown in Table 1 may be measured as the urinary tumor marker. Namely, the compound whose mass is measured as 197.04555 is measured in the LC/MS negative ion detection mode.

In one embodiment, S-adenosylhomocysteine shown in Table 1 may be measured as the urinary tumor marker. Namely, the compound whose mass is measured as 383.11431 is measured in the LC/MS negative ion detection mode.

In one embodiment, 3-methoxy-4-hydroxyphenylglycol shown in Table 1 may be measured as the urinary tumor marker. Namely, the compound whose mass is measured as 167.07028 is measured in the LC/MS positive ion detection mode.

In one embodiment, dopamine shown in Table 1 may be measured as the urinary tumor marker. Namely, the compound whose mass is measured as 154.08626 is measured in the LC/MS positive ion detection mode.

In one embodiment, cystathionine shown in Table 1 may be measured as the urinary tumor marker. Namely, the compound whose mass is measured as 223.07471 is measured in the LC/MS positive ion detection mode.

The mass spectrometer used in the analysis of the metabolites shown in Table 1 has a very high resolution, thus, the mass can be measured to 2, 3, 4, or 5 digits after the decimal point, but when using a mass spectrometer having a low resolution, the integer mass or the mass of one digit after the decimal point may be measured.

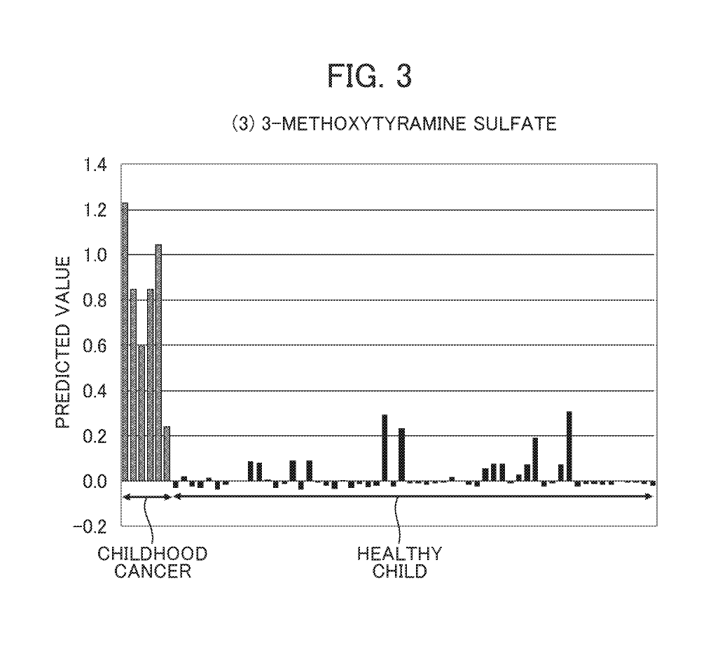

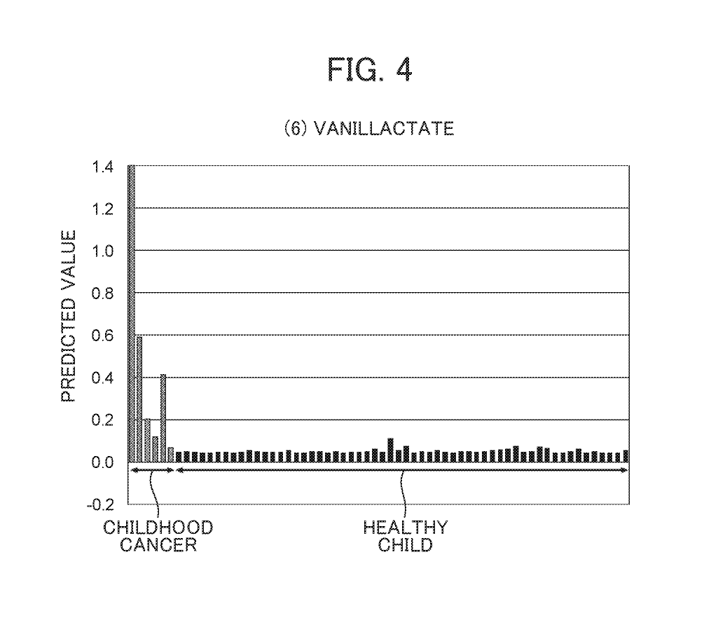

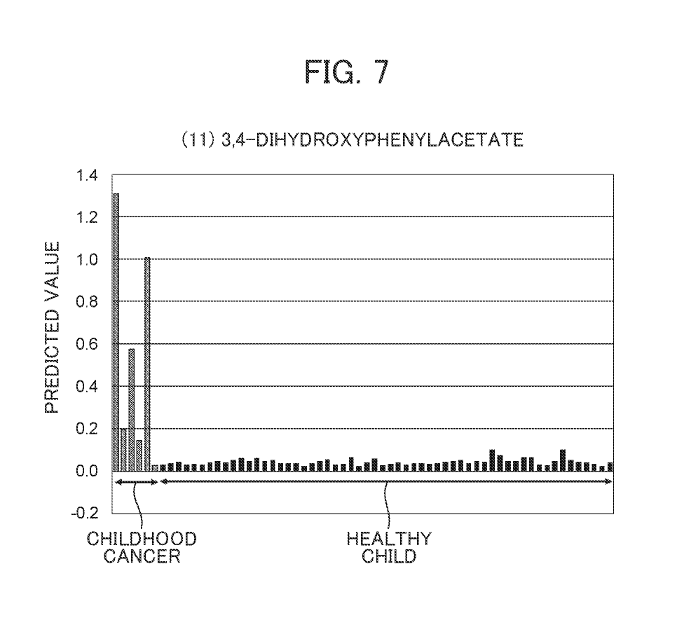

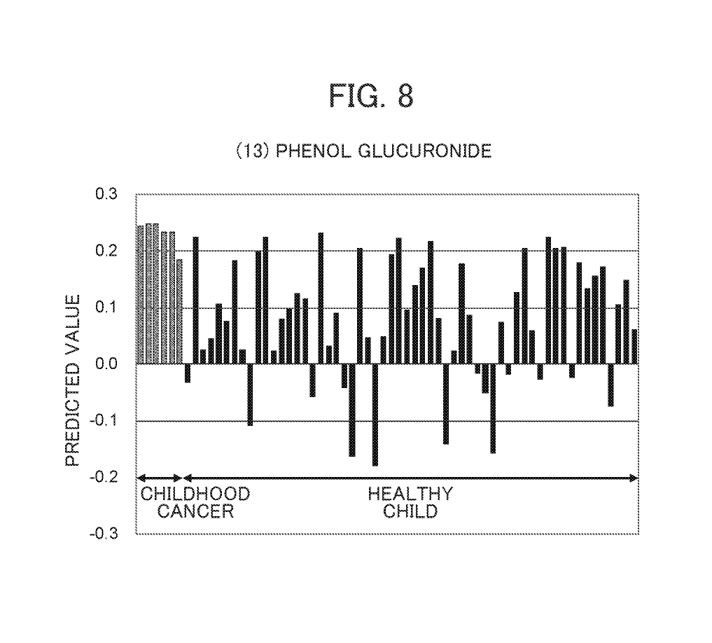

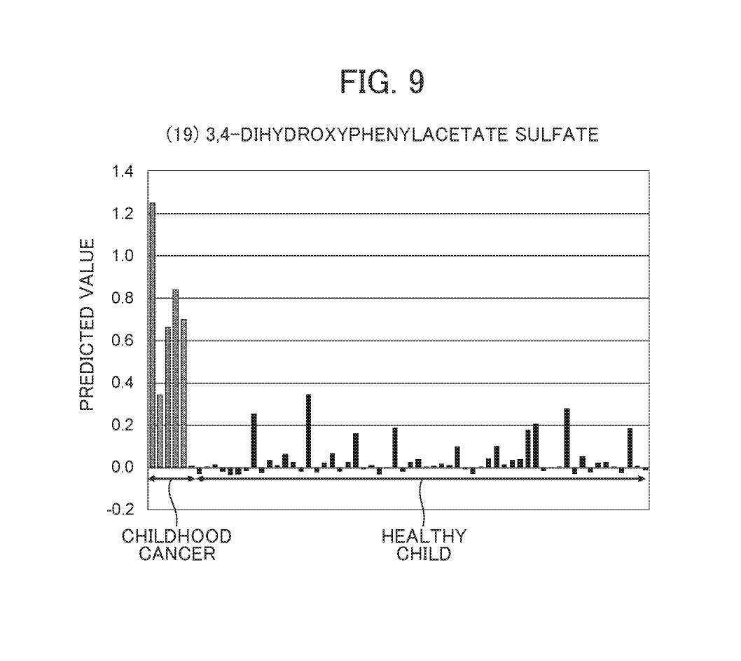

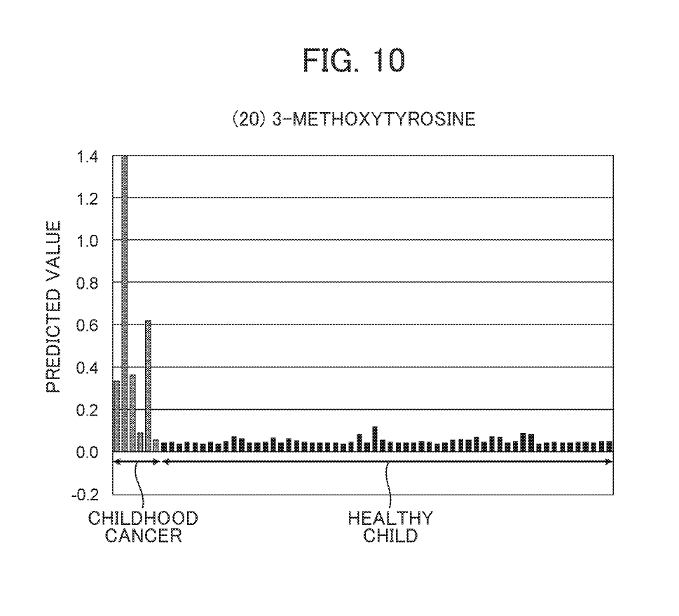

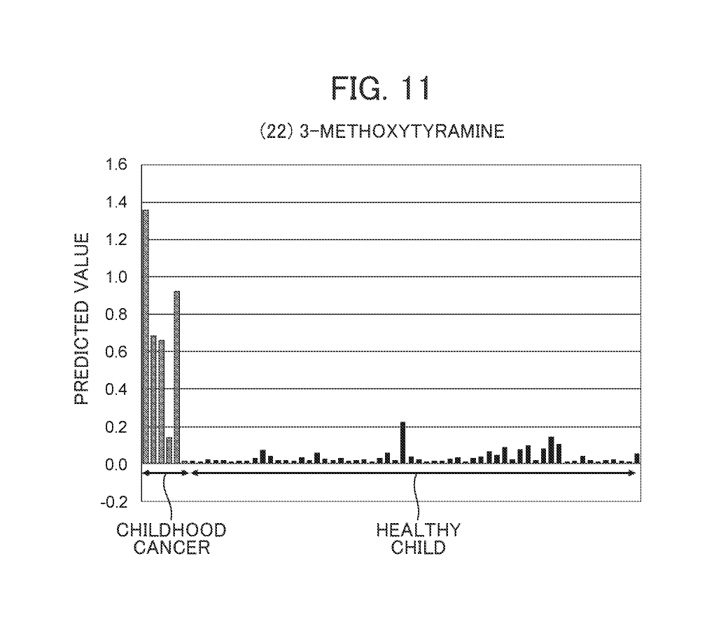

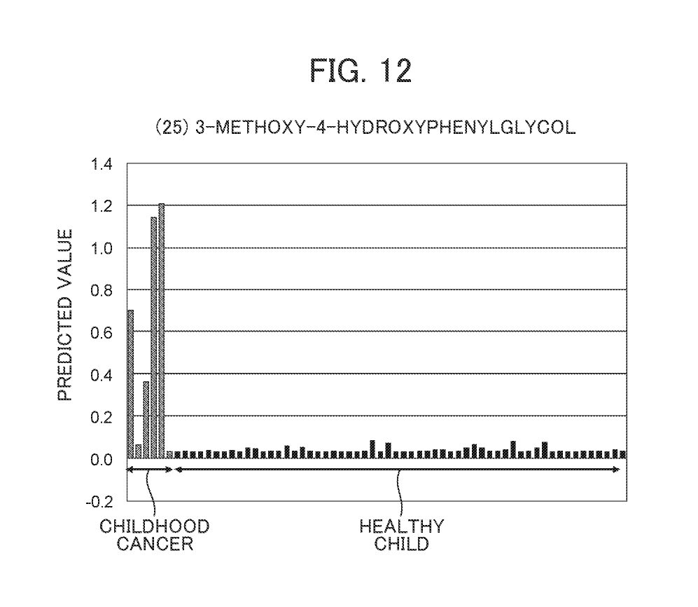

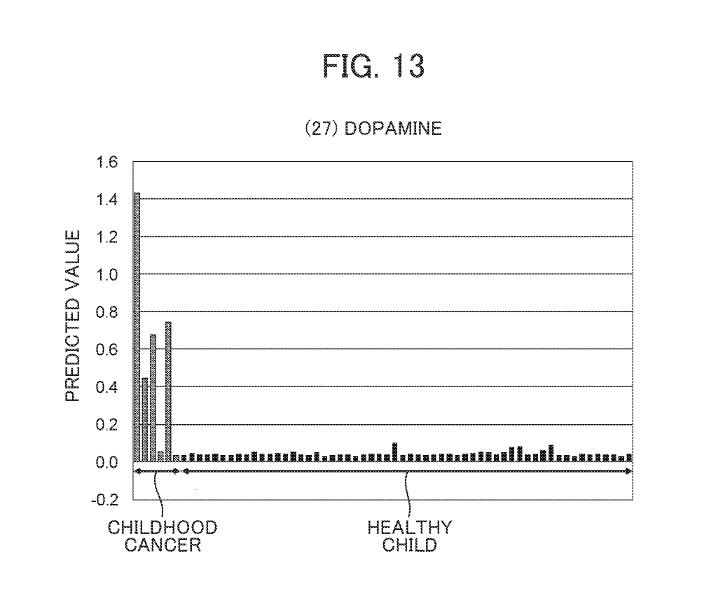

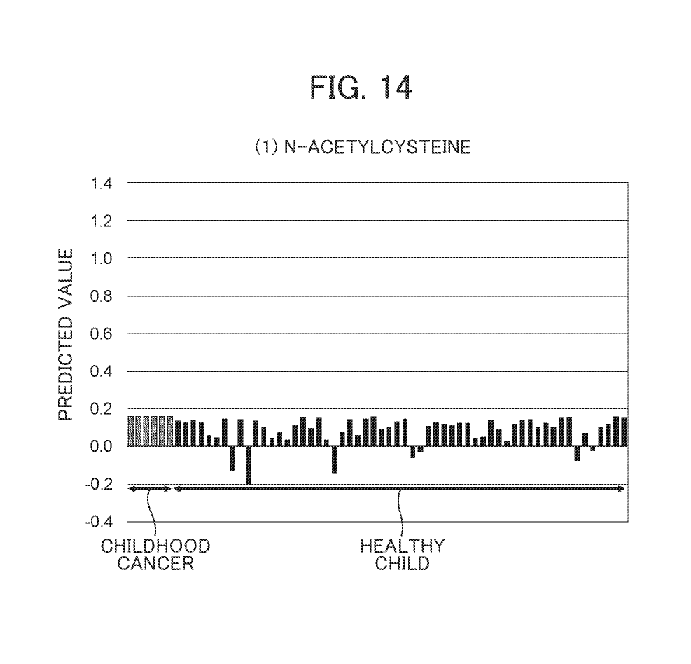

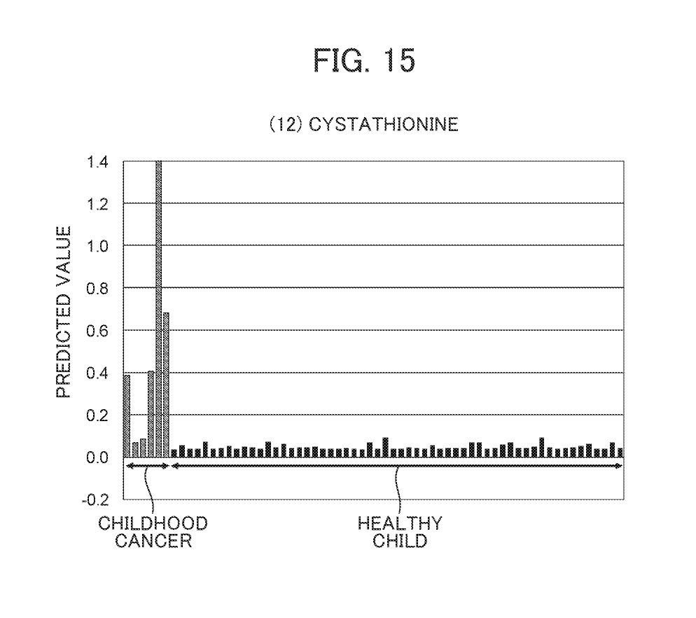

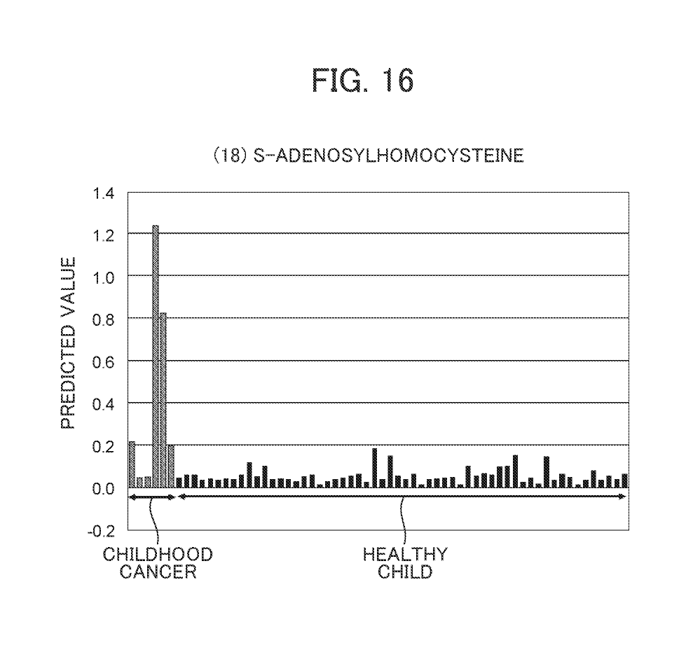

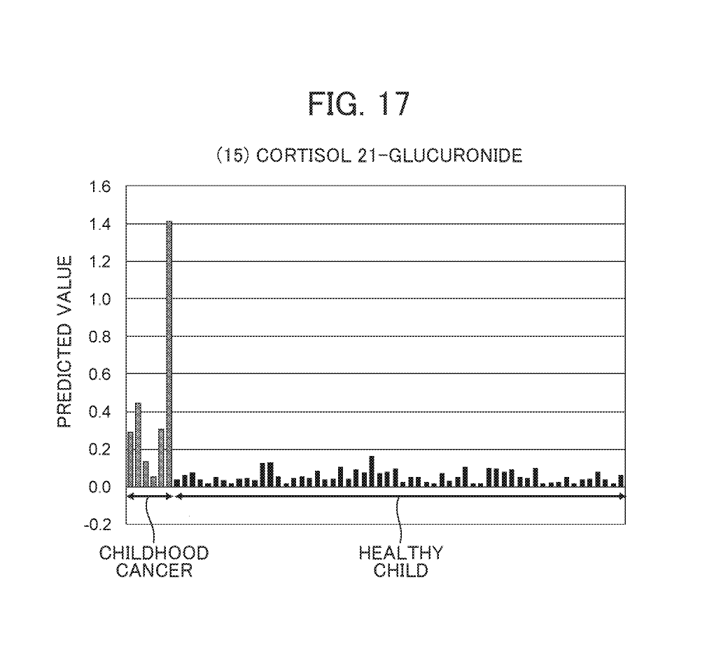

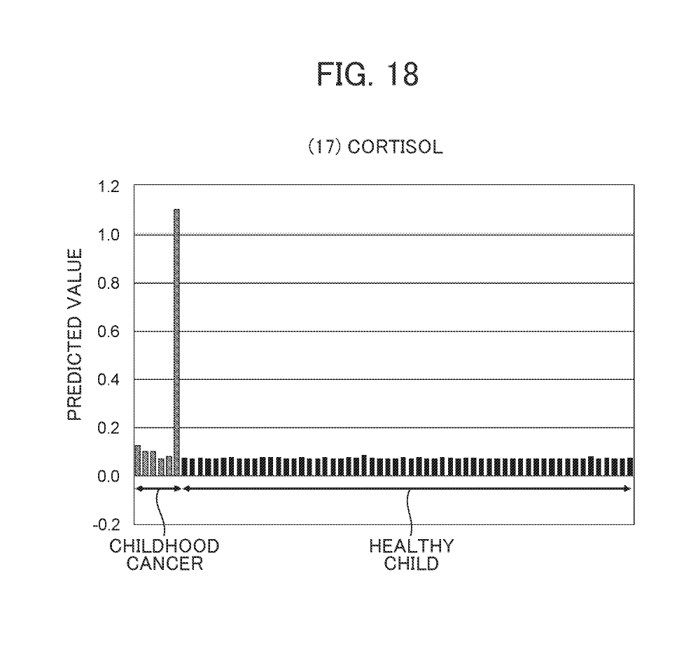

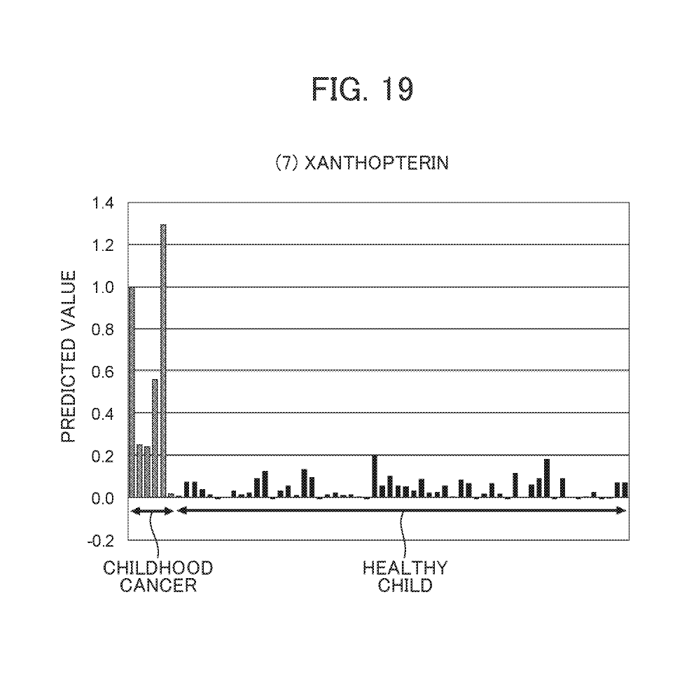

In the present invention, one of the urinary tumor markers shown in Table 1 can be used to evaluate the childhood cancer and monitor the effect of the treatment. The single regression analysis results for these metabolites are shown in FIGS. 3 to 19. Specifically, at least one metabolite selected from the group consisting of:

(i) a metabolite of the tyrosine metabolic pathway selected from the group consisting of 3-methoxytyramine sulfate, vanillactate, homovanillate, vanillylmandelate, 3,4-dihydroxyphenylacetate, phenol glucuronide, 3,4-dihydroxyphenylacetate sulfate, 3-methoxytyrosine, 3-methoxytyramine, 3-methoxy-4-hydroxyphenylglycol, and dopamine;
(ii) a metabolite of the methionine metabolic pathway selected from the group consisting of N-acetylcysteine, cystathionine, and S-adenosylhomocysteine;
(iii) a metabolite of the corticosteroid metabolic pathway selected from the group consisting of cortisol 21-glucuronide and cortisol; and
(iv) a metabolite of the pterin metabolic pathway which is xanthopterin
is measured (provided that if the urinary metabolite to be evaluated is only one, it is not homovanillate or vanillylmandelate).

Further, in the present invention, it is possible to more precisely and accurately evaluate childhood cancers, and monitor the effect of the treatment by using at least two or three or more of the urinary tumor markers in combination. For example, the urinary tumor markers shown in Table 1 can be combined. The combinations of the markers are not specifically limited. In the preferred embodiment, the urinary tumor markers related to different metabolic pathways may be used in combination. Specifically, the combinations are at least two metabolites selected from the group consisting of:

(i) a metabolite of the tyrosine metabolic pathway selected from the group consisting of 3-methoxytyramine sulfate, vanillactate, homovanillate, vanillylmandelate, 3,4-dihydroxyphenylacetate, phenol glucuronide, 3,4-dihydroxyphenylacetate sulfate, 3-methoxytyrosine, 3-methoxytyramine, 3-methoxy-4-hydroxyphenylglycol, and dopamine;
(ii) a metabolite of the methionine metabolic pathway selected from the group consisting of N-acetylcysteine, cystathionine, and S-adenosylhomocysteine;
(iii) a metabolite of the corticosteroid metabolic pathway selected from the group consisting of cortisol 21-glucuronide and cortisol; and
(iv) a metabolite of the pterin metabolic pathway which is xanthopterin,

wherein

a combination of at least one metabolite of the tyrosine metabolic pathway selected from the group of (i) and at least one metabolite of the corticosteroid metabolic pathway selected from the group of (iii);

a combination of at least one metabolite of the tyrosine metabolic pathway selected from the group of (i) and at least one metabolite of the methionine metabolic pathway selected from the group of (ii);

a combination of at least two metabolites of the tyrosine metabolic pathway selected from the group of (i);

a combination of at least one metabolite of the tyrosine metabolic pathway selected from the group of (i) and at least one metabolite of the pterin metabolic pathway selected from the group of (iv);

a combination of at least one metabolite of the corticosteroid metabolic pathway selected from the group of (iii) and at least one metabolite of the pterin metabolic pathway selected from the group of (iv);

a combination of at least one metabolite of the methionine metabolic pathway selected from the group of (ii) and at least one metabolite of the pterin metabolic pathway selected from the group of (iv);

a combination of at least two metabolites of the methionine metabolic pathway selected from the group of (ii);

a combination of at least one metabolite of the methionine metabolic pathway selected from the group of (ii) and at least one metabolite of the corticosteroid metabolic pathway selected from the group of (iii);

a combination of at least two metabolites of the corticosteroid metabolic pathway selected from the group of (iii);

a combination of at least one metabolite of the tyrosine metabolic pathway selected from the group of (i), at least one metabolite of the methionine metabolic pathway selected from the group of (ii) and at least one metabolite of the corticosteroid metabolic pathway selected from the group of (iii);

a combination of at least one metabolite of the tyrosine metabolic pathway selected from the group of (i), at least one metabolite of the methionine metabolic pathway selected from the group of (ii) and at least one metabolite of the pterin metabolic pathway selected from the group of (iv);

a combination of at least one metabolite of the tyrosine metabolic pathway selected from the group of (i), at least one metabolite of the corticosteroid metabolic pathway selected from the group of (iii), and at least one metabolite of the pterin metabolic pathway selected from the group of (iv);

a combination of at least one metabolite of the methionine metabolic pathway selected from the group of (ii), at least one metabolite of the corticosteroid metabolic pathway selected from the group of (iii) and at least one metabolite of the pterin metabolic pathway selected from the group of (iv);

a combination of at least two metabolites of the tyrosine metabolic pathway selected from the group of (i) and at least one metabolite of the corticosteroid metabolic pathway selected from the group of (iii);

a combination of at least two metabolites of the tyrosine metabolic pathway selected from the group of (i) and at least one metabolite of the methionine metabolic pathway selected from the group of (ii);

a combination of at least two metabolites of the tyrosine metabolic pathway selected from the group of (i) and at least one metabolite of the pterin metabolic pathway selected from the group of (iv); and

a combination of at least one metabolite of the tyrosine metabolic pathway selected from the group of (i), at least one metabolite of the methionine metabolic pathway selected from the group of (ii), at least one metabolite of the corticosteroid metabolic pathway selected from the group of (iii) and at least one metabolite of the pterin metabolic pathway selected from the group of (iv) are measured.

A single urinary tumor marker may be evaluated individually, but when examining two or more urinary tumor markers, the combinations thereof are numerous, thus, the evaluation is extremely complicated. Evaluation variables which are a precision variable R2Y and a prediction variable Q2 shown below can be used for two or more combinations as the criteria for determining which combinations are good.

$$R 2 Y = 1 - \frac{\sum {( Yobs - Ycalc )}^{2}}{\sum {( Yobs - \overline{Y} )}^{2}}$$

$$Q 2 = 1 - \frac{\sum {( Yobs - Ypred )}^{2}}{\sum {( Yobs - \overline{Y} )}^{2}}$$

Herein, Yobs represents the measured value, Ycalc represents the calculated value by OPLS, Ypred represents the predicted value when performing a cross validation, and Y̅

represents the mean value. The cross validation is a technique for dividing data, analyzing a part of the data first, then, testing the analysis with the remaining portion of the data, and applying the results to verify and confirm the validity of the analysis itself. Accordingly, the closer the value of the precision variable R2Y is to 1, the higher the accuracy of the model, and the closer the Q2 value of the prediction variable is to 1, the higher the predictability of the model. It is thought that a more accurate evaluation becomes possible by using the combination of the high precision variable and the high prediction variable value in the evaluation of a childhood cancers.

The combination of urinary tumor markers can be appropriately selected in accordance with the type of childhood cancer to be the evaluated, the species, gender, and age of the subject, and the purpose of the evaluation of the childhood cancer.

In a specific embodiment, for example, the following combinations may be measured.

| | Combination example of | |
|---|---|---|
| Number | markers | |
| 1 | 3-methoxytyramine sulfate | cortisol |
| 2 | 3-methoxytyramine sulfate | cortisol 21-glucuronide |
| 3 | 3-methoxytyramine sulfate | N-acetylcysteine |
| 4 | 3-methoxytyramine sulfate | 3-methoxytyrosine |
| 5 | 3-methoxytyramine sulfate | phenol glucuronide |
| 6 | 3-methoxytyramine sulfate | 3,4-dihydroxyphenylacetate |
| 7 | 3-methoxytyramine sulfate | 3,4-dihydroxyphenylacetate sulfate |
| 8 | 3-methoxytyramine sulfate | homovanillate (HVA) |
| 9 | 3-methoxytyramine sulfate | xanthopterin |
| 10 | 3-methoxytyramine sulfate | vanillactate |
| 11 | 3-methoxytyramine sulfate | 3-methoxytyramine |
| 12 | 3-methoxytyramine sulfate | vanillylmandelate (VMA) |
| 13 | 3-methoxytyramine sulfate | S-adenosylhomocysteine (SAH) |
| 14 | 3-methoxytyramine sulfate | 3-methoxy-4-hydroxyphenylglycol |
| 15 | 3-methoxytyramine sulfate | dopamine |
| 16 | homovanillate (HVA) | cortisol 21-glucuronide |
| 17 | 3,4-dihydroxyphenylacetate | cortisol 21-glucuronide |
| | sulfate | |
| 18 | 3-methoxytyramine | cortisol 21-glucuronide |
| 19 | vanillylmandelate (VMA) | 3-methoxytyrosine |
| 20 | 3-methoxytyrosine | 3-methoxy-4-hydroxyphenylglycol |
| 21 | 3-methoxytyramine | cortisol |
| 22 | vanillylmandelate (VMA) | cortisol 21-glucuronide |
| 23 | 3,4-dihydroxyphenylacetate | 3-methoxytyrosine |
| | sulfate | |
| 24 | 3,4-dihydroxyphenylacetate | cortisol |
| | sulfate | |
| 25 | homovanillate (HVA) | 3-methoxytyrosine |
| 26 | vanillylmandelate (VMA) | 3,4-dihydroxyphenylacetate sulfate |
| 27 | 3-methoxytyramine | S-adenosylhomocysteine (SAH) |
| 28 | homovanillate (HVA) | xanthopterin |
| 29 | xanthopterin | cortisol 21-glucuronide |
| 30 | 3,4-dihydroxyphenylacetate | 3-methoxy-4-hydroxyphenylglycol |
| | sulfate | |

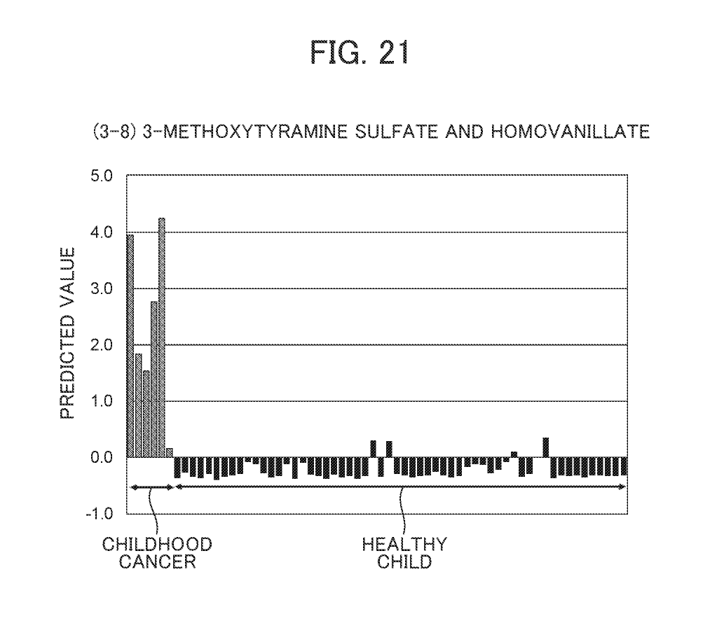

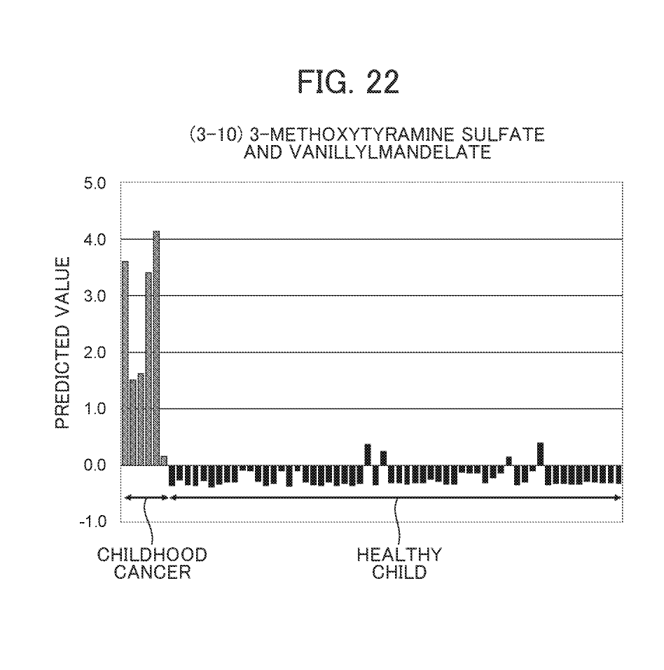

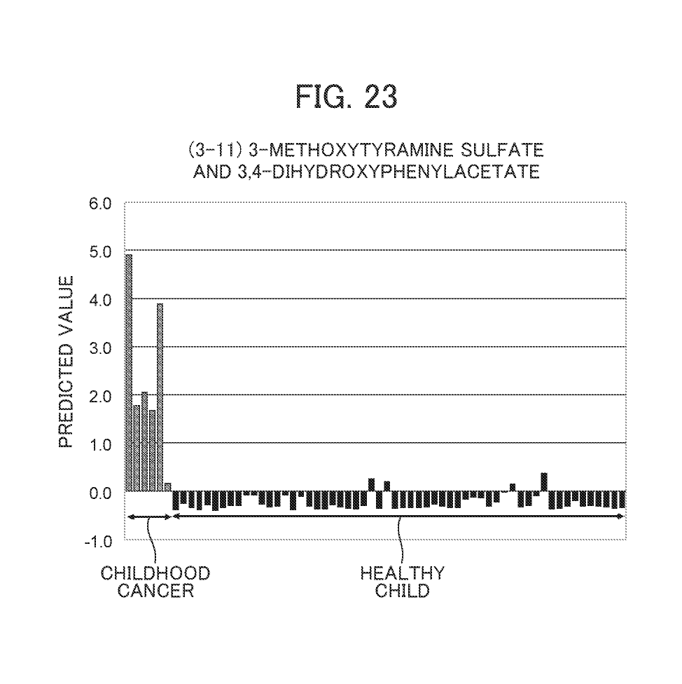

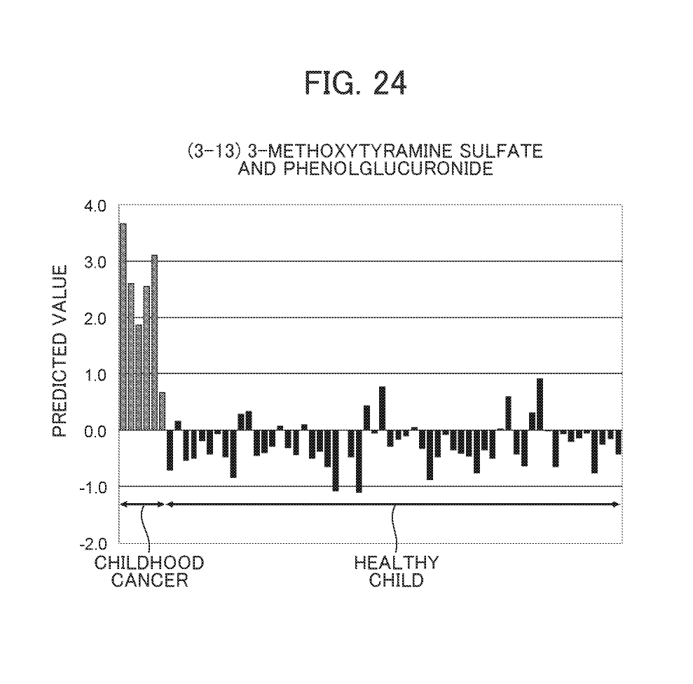

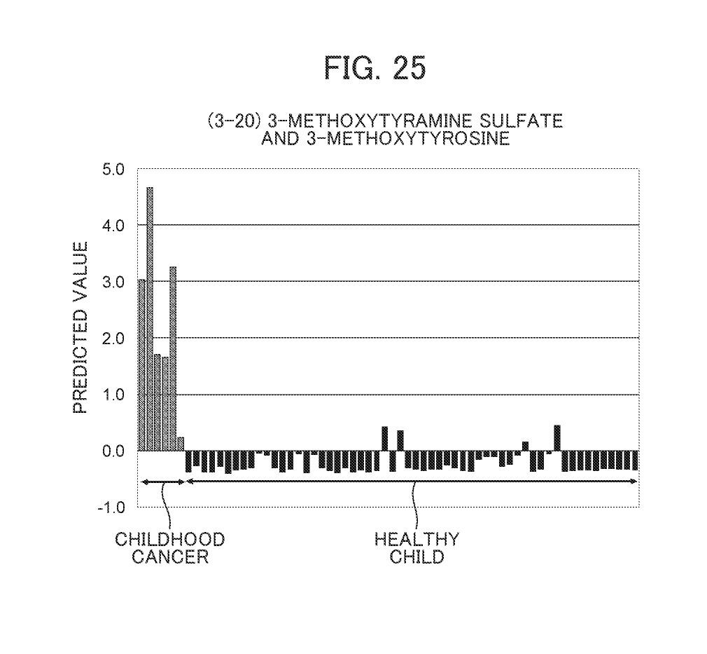

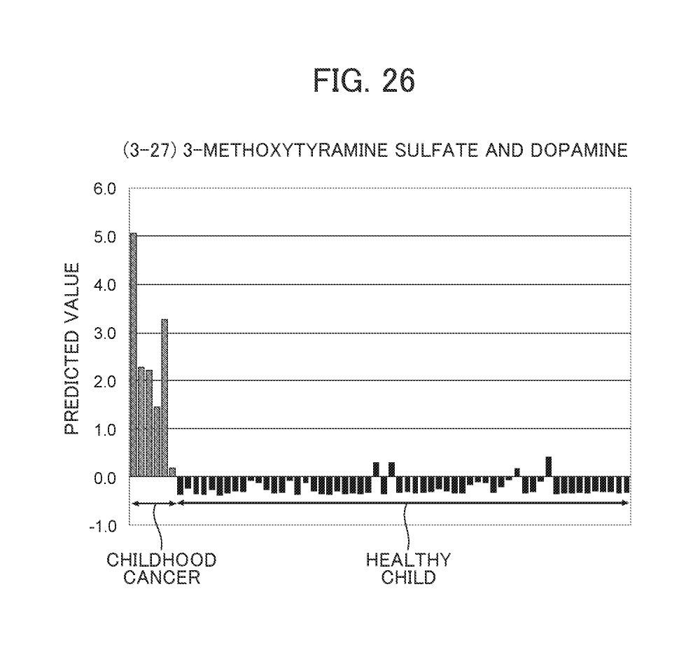

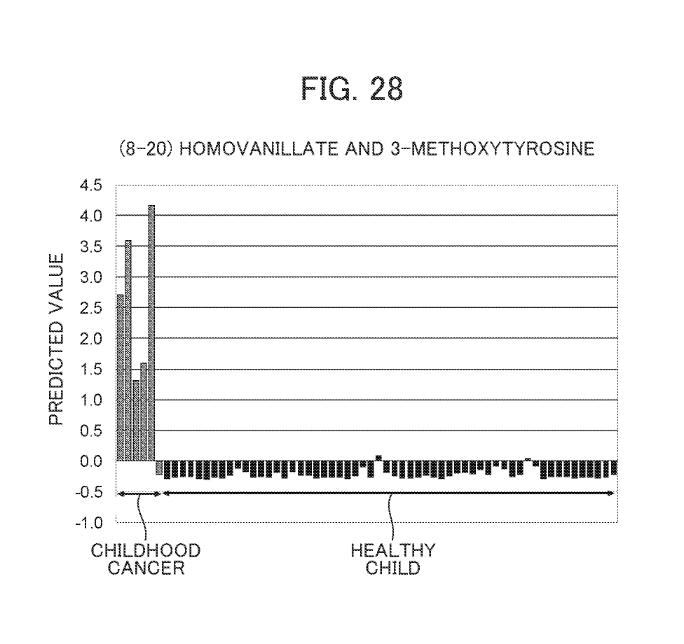

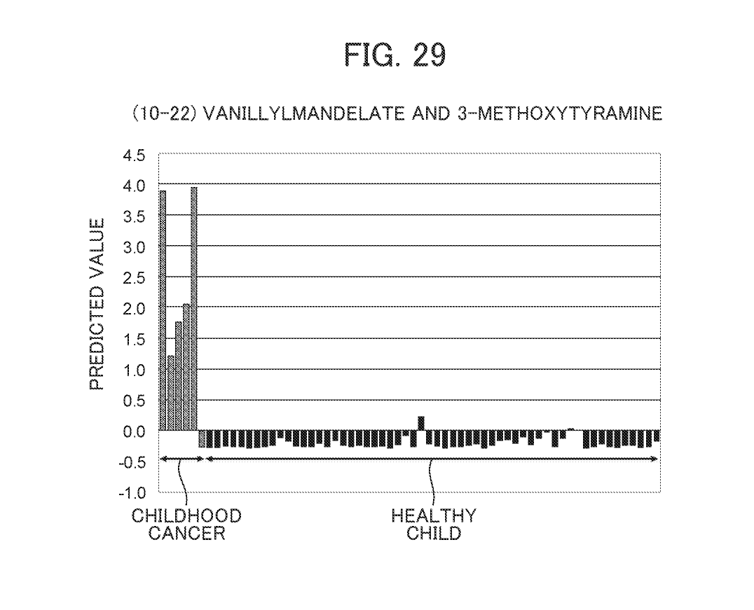

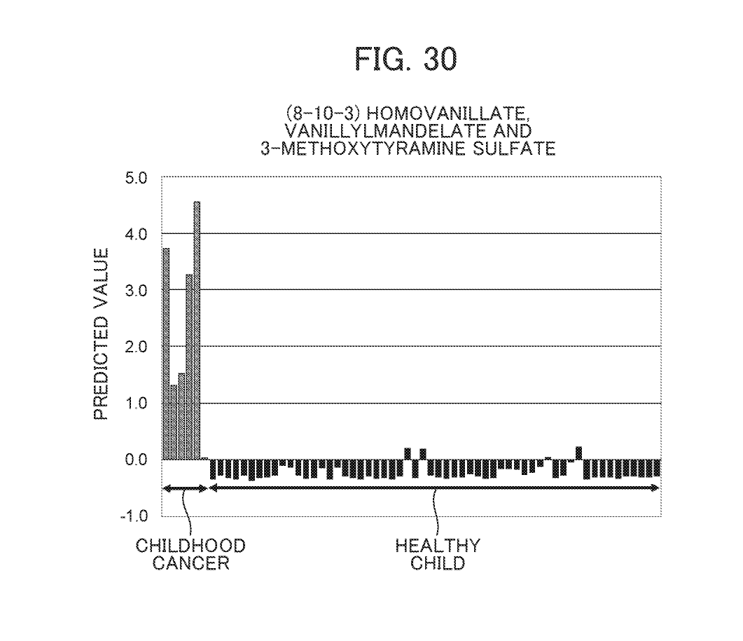

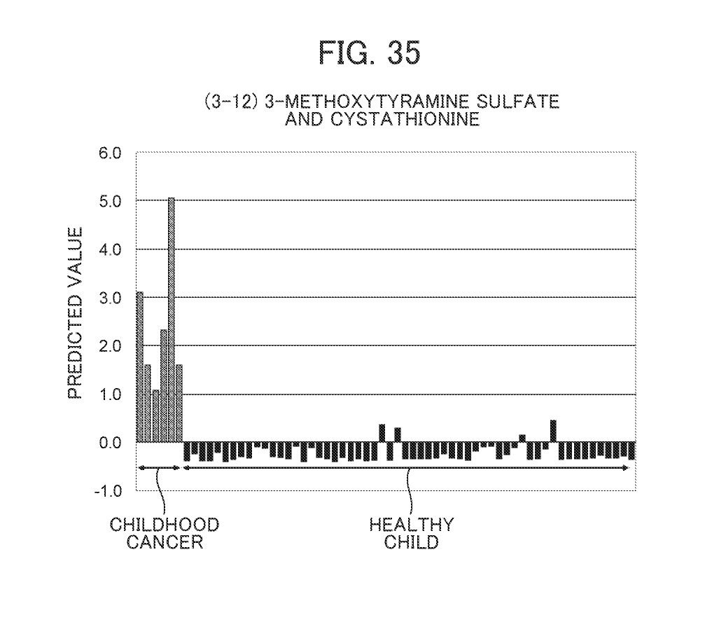

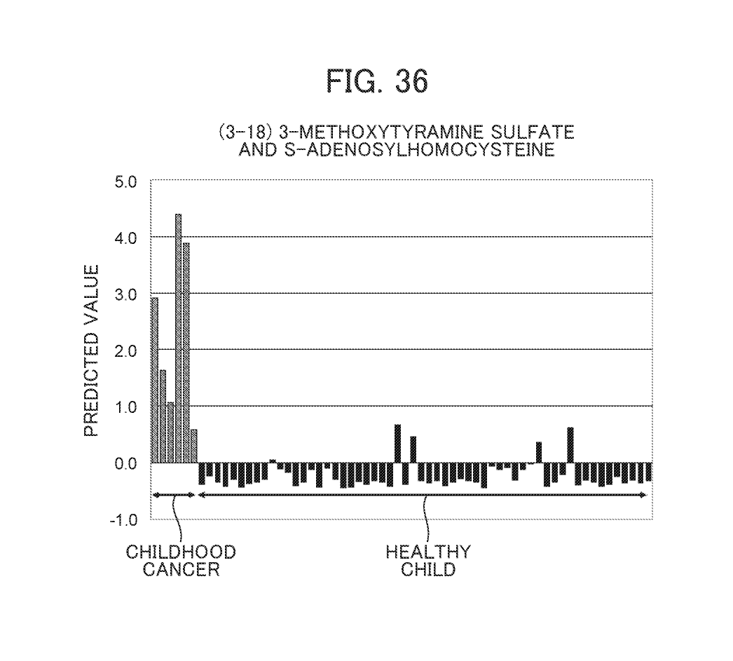

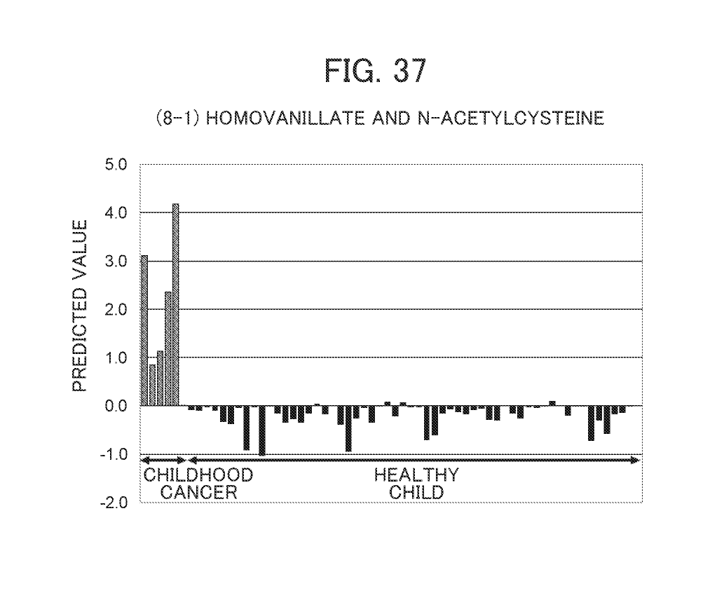

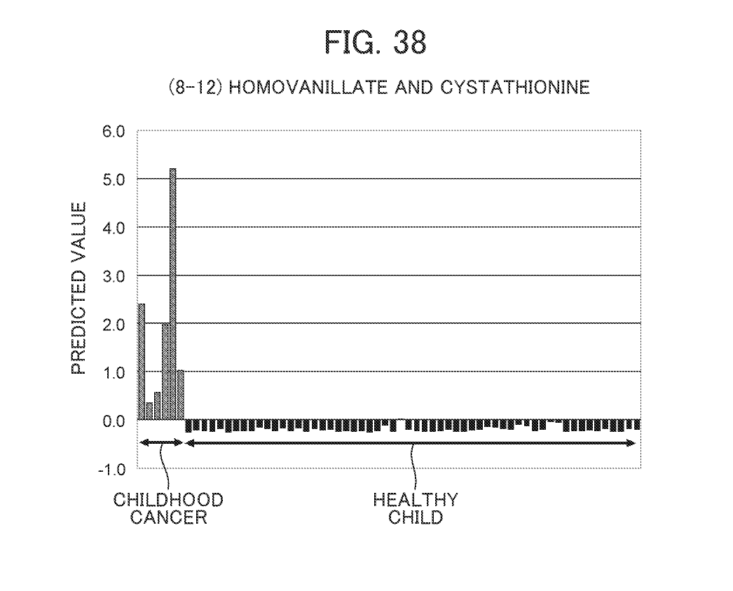

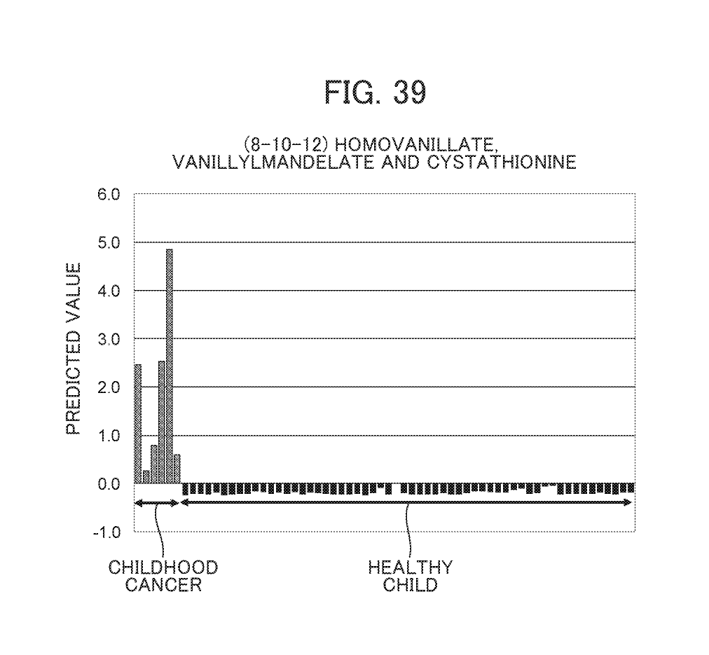

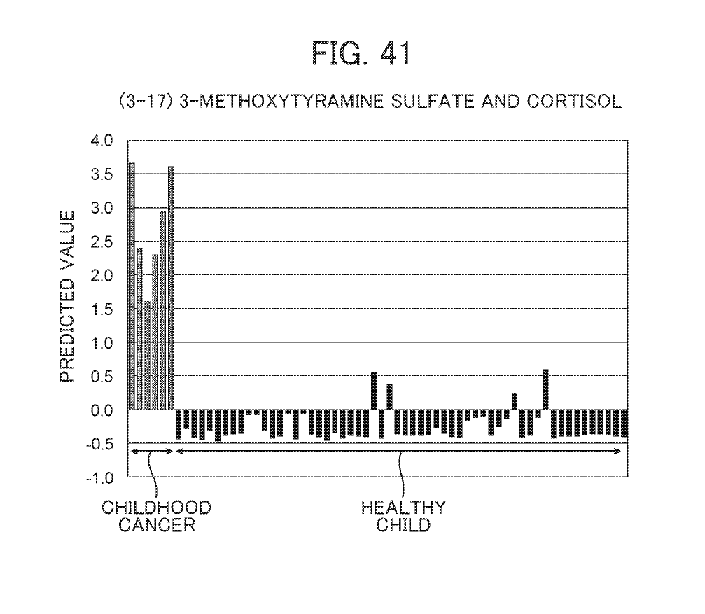

A Partial Least Squares which is one type of multivariate analysis, specifically, OPLS-DA can be used as an example of a method for discriminating the urinary tumor markers. In metabolite analysis, there are cases when the numerous metabolites which vary in cancer patients and healthy individuals are found. It is sometimes difficult to understand the features of the data when using multidimensional data as is, thus, it is preferable to visualize the data by compressing it to two-dimensional or three-dimensional data. For example, FIGS. 3 to 46 show examples of the plotted analysis results. It is possible to use a known analysis method in the technical field such as Principal Component Analysis as the multivariate analysis.

The cancers to be evaluated according to the present invention are childhood cancers. Childhood cancer refers to a malignant tumor afflicting a child, specifically, a child from 0 to 15 years old. Specific examples include leukemia, brain tumor (neuroglioma, germ cell tumor, medulloblastoma, etc.), spinal cord tumor, neuroblastoma, lymphoma, retinoblastoma, malignant bone tumor (osteosarcoma, Ewing sarcoma, etc.), malignant tumors of the kidneys (nephroblastoma, Wilms tumor, etc.), rhabdomyosarcoma, hepatoblastoma, germ cell tumors and the like. Specifically, the present invention may preferably be used in the evaluation of neuroblastoma. Cancers include primary, metastatic and recurrent cancers, and are classified into stages from the degree of progress and the extent of spread. Depending on the differences among primary, metastatic and recurrent cancers and the differences of the stages, the necessary treatments (treatment methods) are also different.

A urine sample means the urine collected from a subject, or a sample obtained by processing the urine (for example, urine to which preservatives such as toluene, xylene or hydrochloric acid are added).

Further, the subject is a child, specifically, a child from 0 to 15 years old. However, the subject is not limited to human beings, and other mammals, for example, primates (monkeys, chimpanzees, etc.), livestock (cows, horses, pigs, sheep, etc.), pets (dogs, cats, etc.), research animals (mice, rats, rabbits, etc.) and the like may be used.

The measurement of the urinary tumor markers means, preferably, the semi-quantitative or quantitative measurement of the amount or the concentration in a urine sample, and the amount may be an absolute amount or may be a relative amount. The measurement can be conducted directly or indirectly. The direct measurement includes measuring the amount or the concentration based on a signal directly correlated with the number of molecules of the urinary metabolites present in the sample. Such signal is based on, for example, the particular physical or chemical properties of the urinary metabolites. The indirect measurement is a measurement of the signal obtained from a secondary component (i.e., a component other than a urinary metabolite), for example, a ligand, a label, or an enzyme reaction product.

In one embodiment of the present invention, the urinary tumor marker, i.e., the urinary metabolite is measured, and this measurement method can use a method or means known in the technical field, and is not specifically limited. For example, the measurement of the urinary tumor marker can be performed by means for measuring the particular physical or chemical properties in the urinary metabolite, for example, means for measuring the accurate molecular weight or the NMR spectrum. Examples of the means for measuring a urinary metabolite include an analyzing apparatus such as a mass spectrometer, an NMR analyzer, a two-dimensional electrophoresis apparatus, a chromatography apparatus, and a liquid chromatography mass spectrometer (LC/MS). These analyzing apparatuses may be used alone to measure the urinary tumor markers, or a plurality of analyzing apparatuses may be combined to measure the urinary tumor markers.

Alternatively, when a reagent for detecting a metabolite to be measured, for example, an immunoreaction reagent, an enzyme reaction reagent or the like can be used, and the urinary metabolite can be measured using such a reagent.

The urinary metabolites shown in Table 1 are found by the LC/MS, thus, these urinary metabolites can be measured using the LC/MS.

As stated above, the urinary tumor marker contained in the urine sample collected from the subject can be measured, and the childhood cancer in the subject can be evaluated based on the result. Furthermore, the urinary tumor markers in the urine samples collected from the subject at a plurality of times may be measured.

The presence and progression of a childhood cancer can be determined at an early stage by the evaluation of the childhood cancer method of the present invention. Namely, the present invention enables determination of the presence or the absence of a childhood cancer at an early stage, which cannot be recognized by currently available diagnostic techniques or criteria, and can predict the malignancy and the prognosis of the childhood cancer. Therefore, the subject can receive the treatment for the childhood cancer at an early stage, and can receive a treatment suitable for the specific malignancy. Further, the effect of the treatment of the childhood cancer can be monitored, and it is possible to consider the termination, continuation or change of the treatment according to the effect of the treatment. Furthermore, as a urine sample is used, the present invention is minimally invasive, can evaluate childhood cancers conveniently and at a low cost, and has a particularly great advantage for children who have difficulty with regular blood sampling.

The method for evaluating a childhood cancer of the present invention can be easily and conveniently performed using a kit and/or a device provided with a means for measuring a urinary tumor marker which is a urinary metabolite.

The kit for the evaluation of a childhood cancer according to the present invention includes at least one of the following means:

a means for measuring a urinary metabolite(s) in a urine sample, preferably at least one of the urinary metabolites shown in Table 1 above.

An example of the kit of the present invention may be a reagent set for mass spectrometry, which contains, for example, an isotope labeling reagent, a fractionating minicolumn, a buffer solution and the like. Another example of the kit may be an immunoreaction reagent set, which contains, for example, a substrate on which a primary antibody is immobilized, a secondary antibody and the like. A further example of the kit may be an enzymatic reaction reagent set, which contains, for example, enzymes, a buffer and the like. The kit of the present invention may include instructions describing the procedures and protocols for carrying out the method of the present invention, and tables showing the reference values or reference ranges for use in the evaluation of a childhood cancer and the like.

The components contained in the kit of the present invention may be provided individually or may be provided in a single container. Preferably, the kit of the present invention includes all of the components necessary for carrying out the method of the present invention, such that these components can be immediately used, for example, with their concentrations adjusted.

A device for evaluating a childhood cancer according to the present invention is provided with the followings:

a measurement unit which is configured to measure a urinary metabolite(s) in a urine sample, preferably at least one urinary metabolite shown in Table 1 above,

a comparison unit which is configured to compare a measurement value of the urinary metabolite measured by the measurement unit with a reference value or a previous measurement value, and

a determination unit which is configured to evaluate the childhood cancer from a comparison result obtained by the comparison unit.

Further, a device for evaluating a childhood cancer according to the present invention, when using a multivariate analysis, is provided with the followings:

a measurement unit which is configured to measure a urinary metabolite(s) in a urine sample, preferably at least two of the urinary metabolites shown in Table 1 above,

a comparison unit which is configured to compare a calculated value (indication showing healthy or childhood cancer) of the objective variable calculated based on the cancer examination model obtained by a multivariate analysis from an explanatory variable (the amount or the concentration of the urinary metabolite, or the intensity ratio of the observed ions of the metabolites which increases/decreases in childhood cancer patients relative to the healthy children) measured by the measurement unit with a reference value or a previous calculated value of the objective variable, and

a determination unit which is configured to evaluate the childhood cancer from a comparison result obtained by the comparison unit.

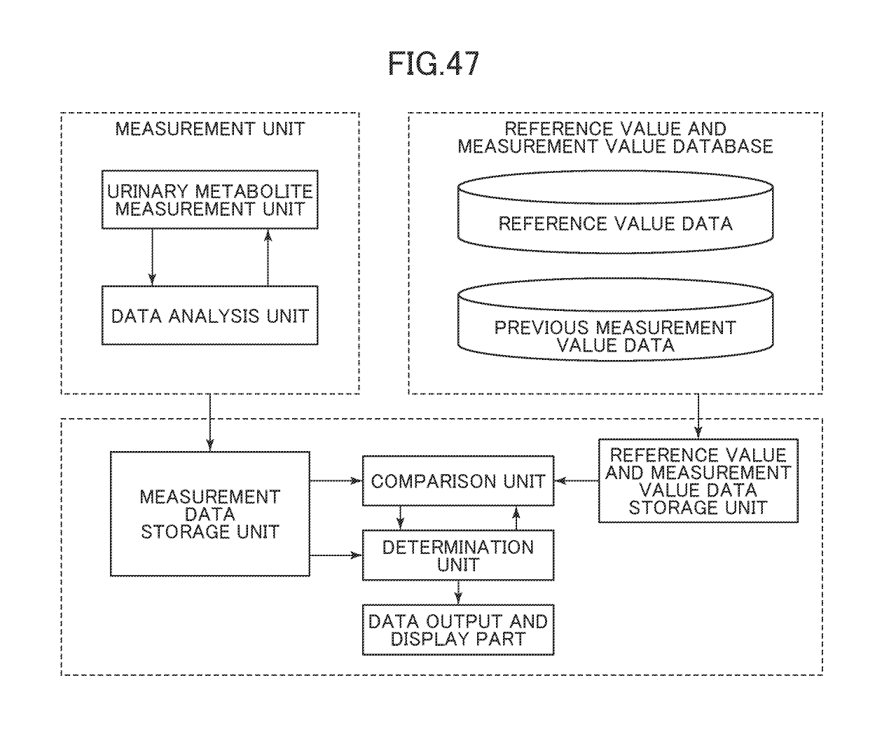

The device of the present invention may preferably be a system in which the measurement unit, the comparison unit and the determination unit are operatively connected to each other so that the method of the present invention can be implemented. An embodiment of the device of the present invention is shown in FIG. 47.

As stated above, the measurement unit includes a means for measuring a urinary metabolite(s) in a urine sample, and is provided with an analyzing apparatus such as a mass spectrometer, an NMR analyzer, a two-dimensional electrophoresis apparatus, a chromatography apparatus, and a liquid chromatography mass spectrometer (LC/MS).

The measurement unit includes a data analysis unit composed of software and a calculator for processing the measurement values obtained from the analyzing apparatus or the like. The data analysis unit calculates the amount or the concentration of the urinary metabolites in a urine sample with reference to data such as a calibration curve on the basis of the measurement values obtained from the analyzing apparatus or the like. On the other hand, the data analysis unit, in the case when a multivariate analysis is used, calculates the calculated value (indication showing healthy or childhood cancer) of the objective variable calculated based on the cancer examination model obtained by the multivariate analysis from the explanatory variable (the amount or the concentration of the urinary metabolite, or the intensity ratio of the observed ions of the metabolites which increases/decreases in childhood cancer patients relative to the healthy children) measured by the measurement unit. The data analysis unit can include, for example, a signal display part, a unit for analyzing the measurement values, a computer unit and the like.

Further, the comparison unit reads out a reference value relating to the amount or the concentration of the urinary metabolite from a storage device (database), and compares the measurement value of the urinary metabolite measured by the measurement unit with the reference value. On the other hand, the comparison unit, when using the multivariate analysis, reads out the reference value of the objective variable from the storage device (database), and compares the calculated value of the objective variable obtained from the measurement unit with the reference value. At this time, the comparison unit selects and reads out a suitable reference value in accordance with the type of urinary tumor marker. Alternatively, when monitoring the same subject over time, the comparison unit reads out the previous measurement value(s) from a storage device (database), and compares it with the measurement value of the urinary metabolite measured by the measurement unit.

Furthermore, the determination unit evaluates the childhood cancer based on the result which compares the measurement value of the urinary metabolite with the reference value in the comparison unit, or based on the result which compares the measurement value of the urinary metabolite at a plurality of times in the comparison unit. On the other hand, the determination unit, when using the multivariate analysis, evaluates the childhood cancer based on the result which compares the calculated value of the objective variable in the comparison unit with the reference value, or based on the result which compares the calculated value of the objective variable at a plurality of times in the comparison unit. The determination unit acquires the information indicating the presence of a childhood cancer and the type of childhood cancer in the subject. A preferable apparatus may be an apparatus that can be used without the knowledge of an experienced clinician, and for example, may be an electronic device in which the sample may be simply added.

The device of the present invention may be further provided with a data storage unit, a data output and display part and the like.

As used herein, “the evaluation of a childhood cancer” means detecting a childhood cancer in a subject, predicting the risk of a childhood cancer in a subject, determining the stage of a childhood cancer in a subject, determining the prognosis of a childhood cancer in a subject, and monitoring the effect of a treatment for a childhood cancer present in a subject. As the treatments to be applied differ in accordance with the malignancy of the childhood cancer, for example, the stage and the prognosis (metastasis, recurrence, etc.), it may be important to determine the stage and the prognosis of the childhood cancer. Further, in the present invention, the “evaluation” also includes the continual monitoring of an already evaluated or diagnosed childhood cancer, and the verification of an already conducted evaluation or diagnosis of a childhood cancer.

The “evaluation” in accordance with the method, the kit and the device for evaluating a childhood cancer according to the present invention is intended to be able to evaluate a statistically significant ratio of subjects. Therefore, the “evaluation” by the method, the kit, and the device for evaluating a childhood cancer according to the present invention may not always produce correct results for all (i.e., 100%) subjects of the evaluation. The statistically significant ratio can be determined by use of various well-known statistical evaluation tools, for example, the determination of a confidence interval, the determination of a p-value, Student's t-test, or the Mann-Whitney test. The confidence interval may preferably be at least 90%. The p-value may preferably be 0.1, 0.01, 0.005, or 0.0001. More preferably, the method, the kit, and the device for evaluating a childhood cancer according to the present invention can properly evaluate at least 60%, at least 80%, or at least 90% of the subjects.

A specific example of the evaluation of a childhood cancer is as follows. In one embodiment, a urinary tumor marker (urinary metabolite) in a urine sample of a subject is measured and the measurement value is compared with a reference value.

The reference value is the amount or concentration, or the range of the amount or the concentration of the urinary metabolite, which is an indication of the presence of the specific childhood cancer. On the other hand, when using the multivariate analysis, the calculated value of the objective variable for discriminating healthy children from childhood cancer patients is the reference value. For example, the reference value can be derived from the healthy children (group) or the children (group) with a low childhood cancer risk. Alternatively, the reference value can be derived from patients (patient groups) having a specific childhood cancer, or having a childhood cancer at a known stage, or having a childhood cancer showing a specific prognosis. The reference value applied to individual subjects may be changed in accordance with various physiological parameters such as the species, age and gender of the subject animal.

Preferably, the correlation between the amount or the concentration of the urinary tumor marker and the presence of the specific childhood cancer and/or the specific stage or the prognosis of the childhood cancer may be recorded in a database. Moreover, the measurement value of the urinary tumor marker in the measured urine sample can be compared with the reference value in the database. This kind of database is useful as the reference value or the reference range which is an indication of childhood cancer, specifically, the presence or absence of a childhood cancer, the specific stage or the prognosis.

The amount or concentration of the urinary metabolites shown in Table 1 are different in the childhood cancer patients and the healthy children, and the amount or the concentration may change depending on the presence of a childhood cancer as well as prior to the start of or after the start of treatment. For example, among the urinary tumor markers shown in Table 1, the amount or the concentration of phenol glucuronide and N-acetylcysteine decreases in childhood cancer patients compared with the healthy children, and the amount or the concentration of 3-methoxytyramine sulfate, vanillactate, homovanillate, vanillylmandelate, 3,4-dihydroxyphenylacetate, 3,4-dihydroxyphenylacetate sulfate, 3-methoxytyrosine, 3-methoxytyramine, 3-methoxy-4-hydroxyphenylglycol, dopamine, cystathionine, S-adenosylhomocysteine, cortisol 21-glucuronide, cortisol and xanthopterin increases in the childhood cancer patients.

Therefore, in case where the reference value is derived from the healthy children (group) or the children (group) with a low childhood cancer risk, it is indicated that the possibility that the subject will develop a childhood cancer is low when the metabolites for which the amount or the concentration of the urinary metabolite are known to increase in relation to the presence of a childhood cancer are used as the markers, and when the amount or the concentration of the urinary metabolites is equivalent to or lower than the reference value, and to the contrary, the possibility that the subject will develop a childhood cancer is high when the amount or the concentration of the urinary metabolites is higher than the reference value. On the other hand, it is indicated that the possibility that the subject will develop a childhood cancer is low when the metabolites for which the amount or the concentration of the urinary metabolite are known to decrease in relation to the presence of a childhood cancer are used as markers, and when the amount or the concentration of the urinary metabolites is equivalent to or higher than the reference value, and to the contrary, the possibility that the subject will develop a childhood cancer is high when the amount or the concentration of the urinary metabolites is lower than the reference value.

In case where the reference value is derived from the patients (patient group) having a specific childhood cancer, or having a childhood cancer at a known stage, or having a childhood cancer showing a specific prognosis, it is indicated that the possibility that the subject has developed the specific childhood cancer is high, or the possibility of developing the childhood cancer at a known stage is high, or the possibility of showing a specific prognosis is high when the metabolites for which the amount or the concentration of the urinary metabolite are known to increase in relation to the presence of a childhood cancer are used as the markers, the amount or the concentration of the urinary tumor marker is equivalent to or is not significantly different from the reference value, or when the amount or the concentration of the urinary tumor marker is higher than the reference value. On the other hand, it is indicated that the possibility that the subject has developed the specific childhood cancer is high, the possibility of developing the childhood cancer at a known stage is high, or the possibility of showing a specific prognosis is high when the metabolites for which the amount or the concentration of the urinary metabolites are known to decrease in relation to the presence of a childhood cancer are used as the markers, the amount or the concentration of the urinary tumor marker is equivalent to or is not significantly different from the reference value, or when the amount or the concentration of the urinary tumor marker is lower than the reference value. Furthermore, it is also possible to perform a multivariate analysis using a plurality of the urinary tumor markers.

In another embodiment, urine samples may be collected from the subject at a plurality of points in time, the urinary tumor marker(s) contained in the urine sample may be measured at the respective measurement points in time, and the measurement values of the urinary tumor marker(s) may be compared with the respective measurement points in time. More specifically, the amount or the concentration of a urinary tumor marker (a) at a first point in time is compared with the amount or the concentration of a urinary tumor marker (b) at a second point in time. When a multivariate analysis is performed, for example, the calculated value at the first point in time of one component is compared with the calculated value at the second point in time. The measurements can be performed at least 2, 3, 4, 5, 10, 15, 20, 30, or more times over time at an interval of, for example, 1 day, 2 days, 5 days, 1 week, 2 weeks, 3 weeks, 1 month, 2 months, 3 months, 6 months, 1 year, 2 years, 3 years, 5 years, or longer. By this comparison, monitoring over time can be performed, and the progression of the childhood cancer, the metastasis or recurrence, etc., of the childhood cancer can be evaluated.

Further, in another embodiment, the urinary tumor marker used in the present invention can be used to monitor the effect of a treatment (therapeutic drugs or therapeutic methods) on a childhood cancer in a subject. Specifically, the monitoring includes:

(a) a step of measuring a urinary metabolite(s) in a urine sample from a patient having a childhood cancer prior to being treated with a therapeutic drug or a therapeutic method,

(b) a step of measuring the urinary metabolite(s) in a urine sample from the patient having a childhood cancer after being treated with the therapeutic drug or the therapeutic method,

(c) a step of repeating (b) in accordance with need, and

(d) a step of monitoring the effect of the therapeutic drug or the therapeutic method on the childhood cancer on the basis of the measurement results of (a) to (c).

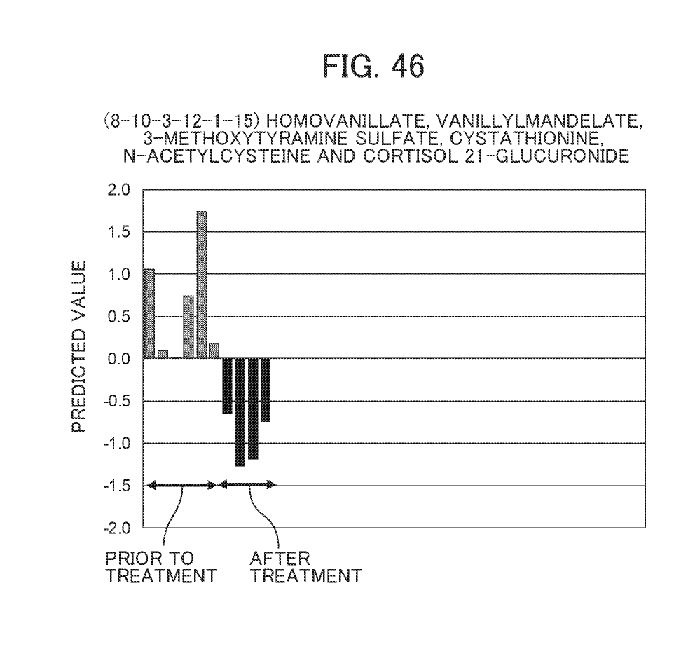

In the aforementioned method, the urine sample may be collected from a patient having a childhood cancer prior to treatment with a therapeutic drug or therapeutic method, and the urinary tumor marker in the urine sample may be measured. After the patient having a childhood cancer is treated with the therapeutic drug or the therapeutic method, urine samples may be collected at suitable periods of time to measure the urinary tumor markers in the urine sample(s). The urine sample(s) may be collected, for example, immediately after treatment, after 30 minutes, after 1 hour, after 3 hours, after 5 hours, after 10 hours, after 15 hours, after 20 hours, after 24 hours (1 day), after 2 to 10 days, after 10 to 20 days, after 20 to 30 days, and after 1 month to 6 months. The measurement of the urinary tumor marker in the urine sample can be performed in the same manner as above. The urinary tumor markers of the present invention, for example, as shown in FIG. 46, can discriminate cancer patient before and after treatment. Therefore, by measuring the urinary tumor marker(s) before and after treatment, it is possible to monitor the effect of the treatment with the therapeutic drug or the therapeutic method. Based on the effect of the monitoring, it can be considered whether to terminate, continue, or change the treatment.

Furthermore, the method for evaluating a childhood cancer may be combined with other conventionally known diagnostic methods for childhood cancers. Examples of known diagnostic methods for childhood cancers include image examination (for example, ultrasound examination, Computed Tomography (CT), X-ray examination, Magnetic Resonance Imaging (MRI), Positron Emission Tomography (PET), etc.), endoscopic examination, pathological examination by biopsy (bone marrow or cerebral spinal fluid examination), the measurement of cancer markers in the blood and the like.

Based on the aforementioned evaluation effect, a physician can diagnose the childhood cancer of the subject, and can perform a suitable treatment. Namely, the present invention is related to a method for evaluating and treating a childhood cancer in a subject. For example, when the childhood cancer in the subject is evaluated in accordance with the method for evaluating a childhood cancer according to the present invention, and it is determined that the subject has a high possibility of developing a childhood cancer, a treatment for treating the childhood cancer in the subject or for preventing the progression of the childhood cancer is performed. Further, when it is evaluated that the stage of the childhood cancer in the subject has progressed or there is a high possibility that the prognosis of the childhood cancer is poor, the treatment is continued, and a change of the therapeutic method is considered according to need. Alternatively, when it is evaluated that the possibility that a childhood cancer is present in the subject is high, another of the aforementioned childhood cancer diagnostic methods may be performed to verify the presence of cancer. Furthermore, based on the evaluation effect before and after treatment, the effect of the treatment is monitored, and the termination, the continuation or a change of the treatment may be determined.

Childhood cancer can be treated by surgery, radiotherapy, chemotherapy, immunotherapy, proton beam therapy, heavy particle beam therapy and the like alone or in combination. The treatment of the childhood cancer can be appropriately selected by a person skilled in the art with reference to the type, stage, and the malignancy of childhood cancer, the gender, age, and condition of the afflicted child, the responsiveness of the afflicted child to treatment and the like.

A cancer examination at an examination center will be described as an example which utilizes the present invention. In the examination center, guidance for a cancer examination may be provided in response to a request, etc., from the examinee. When the examinee applies for a primary examination, the number of biomarkers for the examination may be selected. For example, 1 to 3 types of metabolites may be provided as the number of biomarkers. This examination can also be used in combination with other biomarkers as examination for all types of cancer (to analyze various cancers at once).

Subsequently, the examination center may provide the examinee with an examination kit necessary for the urine collection. The examination kit may be sent by mail in accordance with need. After receiving the examination kit, the examinee may take or send the specimen to the examination center. The specimen may be kept frozen at approximately −80° C. at the examination center for subsequent examination in accordance with need. The examination center may conduct the primary examination and send the examination results to the examinee.

The examinee may receive the primary examination results, may request a secondary examination in response to the contents, and may receive a more detailed diagnosis. Therefore, it is possible to verify the suspected cancer in the primary examination, and furthermore, specify the site of the cancer and/or the type of cancer.

Further, the urinary tumor marker used in the present invention can be used to evaluate the efficacy of a treatment (therapeutic drug or therapeutic method) of the childhood cancer, or to screen therapeutic drug candidates for the childhood cancer. Specifically, the method for evaluating the efficacy of the treatment of a childhood cancer, or the method for screening therapeutic drug candidates for a childhood cancer includes:

(a) a step of measuring a urinary metabolite(s) in a urine sample from an animal having a childhood cancer treated with a therapeutic drug or a therapeutic method to be tested, and

(b) a step of evaluating the efficacy of the therapeutic drug or the therapeutic method to be tested for a childhood cancer on the basis of the measurement result of (a).

In the method of the present invention, a urine sample may be collected from an animal having a childhood cancer, i.e., an animal which has developed a childhood cancer or an animal having a risk of developing cancer, and the urinary tumor marker(s) in the urine sample may be measured. Preferably, prior to treating with the therapeutic drug or the therapeutic method to be tested, the urine sample may be collected from the animal having a childhood cancer, and the urinary tumor marker(s) in the urine sample may be measured. After an animal having a childhood cancer is treated with the therapeutic drug or the therapeutic method to be tested, the urine sample may be collected at suitable periods of time, and the urinary tumor marker(s) in the urine sample may be measured. For example, the urine samples may be collected immediately after treatment, after 30 minutes, after 1 hour, after 3 hours, after 5 hours, after 10 hours, after 15 hours, 20 hours, after 24 hours (1 day), after 2 to 10 days, after 10 to 20 days, after 20 to 30 days, and after 1 month to 6 months. The measurement of the urinary tumor marker in the urine sample and the evaluation of a childhood cancer can be performed in the same manner as above.

The animal subject may be a human afflicted with a childhood cancer, or may be a childhood cancer model animal (mice, rats, rabbits, etc.). Generally, after the efficacy of a therapeutic drug or a therapeutic method to be tested has been verified in a model animal, an evaluation of the efficacy in humans is performed by, for example, clinical trials and the like.

The types of therapeutic drugs or therapeutic methods to be evaluated or screened are not specifically limited. Examples of the therapeutic drug or the therapeutic method to be tested include any physical factors, specifically, naturally occurring molecules, for example, amino acids, peptides, oligopeptides, polypeptides, proteins, nucleic acids, lipids, carbohydrates (sugars, etc.), steroids, glycopeptides, glycoprotein, proteoglycans and the like; synthetic analogs or derivatives of naturally occurring molecules, for example, peptide mimetics, nucleic acid molecules (aptamers, antisense nucleic acids, double stranded RNA (RNAi), etc.) and the like; molecules which do not occur naturally, for example, low molecular organic compounds (inorganic or organic compound library, or a combinatorial library, etc.) and the like; and mixtures thereof. Further, the therapeutic drug or the therapeutic method may be a single substance, a complex constituted from a plurality of substances, or may be food and diet. Furthermore, the therapeutic drug or the therapeutic method to be tested, in addition to the aforementioned physical factors, may be radiation, ultraviolet waves and the like.

Treatments with therapeutic drugs or therapeutic methods to be tested on animals differ depending on the type of the therapeutic drugs or therapeutic methods, and can be easily determined by a person skilled in the art. For example, the administration conditions such as the dose, the dosage period and the administration route of the therapeutic drug to be tested can be appropriately determined by a person skilled in the art.

Further, the efficacy of the therapeutic drug or the therapeutic method to be tested can be examined under several conditions. Examples of such conditions include the treatment times or periods of the therapeutic drug or the therapeutic method to be tested, the amount (larger and small) thereof, and the number of treatment rounds. For example, a dilution series of the therapeutic drug to be tested can be prepared to thereby establish a plurality of doses.

Furthermore, therapeutic drugs or therapeutic methods may be used in combination when examining additive effects, synergistic effects, etc., of a plurality of the therapeutic drugs or therapeutic methods to be tested.

By measuring the urinary tumor marker(s) in the urine sample collected from an animal after the treatment with the therapeutic drug or the therapeutic method to be tested, and comparing with the amount or the concentration of the urinary tumor marker(s) prior to treatment, it can be evaluated as to whether or not the therapeutic drug or the therapeutic method to be tested is effective in, the disappearance of the childhood cancer, the shrinking of the childhood cancer, the improvement of the condition due to the childhood cancer, or the termination or the reduction of the progression of the childhood cancer.

For example, regarding the urinary metabolite of which the amount or the concentration is decreased in a childhood cancer patient compared to a healthy child, the fact that the measurement value after treatment is higher than the measurement value prior to treatment indicates that the therapeutic drug or the therapeutic method to be tested is effective in the disappearance of the childhood cancer, the shrinking of the childhood cancer, the improvement of the condition due to the childhood cancer, or the termination or the reduction of the progression of the childhood cancer. On the other hand, the fact that the measurement value after treatment is lower than the measurement value prior to treatment or is not significantly different from the measurement value prior to treatment indicates that the therapeutic drug or the therapeutic method to be tested is not effective in the treatment of the childhood cancer.

Further, for example, regarding the urinary metabolite of which the amount or the concentration is increased in a childhood cancer patient, the fact that the measurement value after treatment is lower than the measurement value prior to treatment indicates that the therapeutic drug or the therapeutic method to be tested is effective in the disappearance of the childhood cancer, the shrinking of the childhood cancer, the improvement of the condition due to the childhood cancer, the termination or the reduction of the progression of the childhood cancer. On the other hand, the fact that the measurement value after treatment is higher than the measurement value prior to treatment or is not significantly different from the measurement value prior to treatment indicates that the therapeutic drug or the therapeutic method to be tested is not effective in the treatment of the childhood cancer.

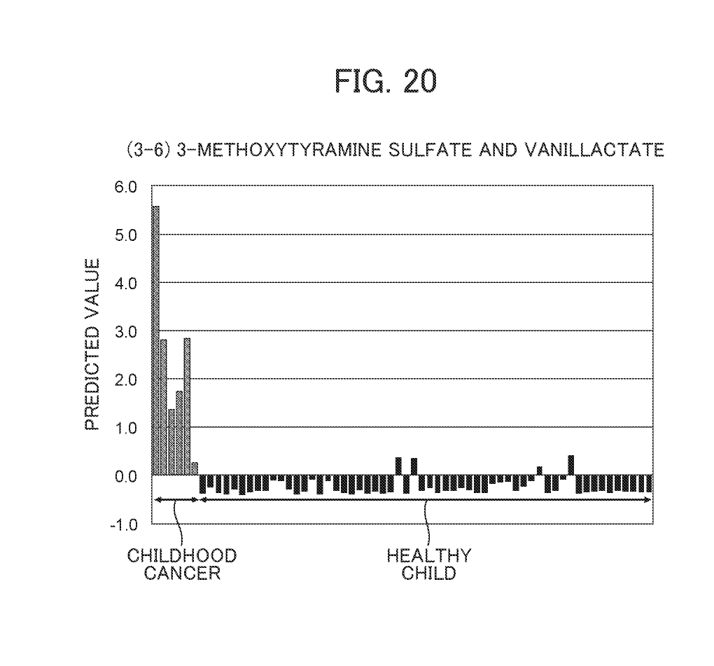

Furthermore, it is possible to carry out a multivariate analysis using these pluralities of urinary tumor markers. For example, when 2, 3, 4, 5, or 6 types of urinary tumor markers are used, the OPLS-DA analysis results could be obtained as shown in FIGS. 20 to 46, but it is understood that a healthy child can be discriminated from a childhood cancer patient by a determination line (for example, the solid line in which the calculated value of the components of the horizontal axis pass through 0). Namely, in the case of a new subject, it is understood that the therapeutic drug or the therapeutic method to be tested is effective in the disappearance of the childhood cancer, the shrinking of the childhood cancer, the improvement of the condition due to the childhood cancer, or the termination or the reduction of the progression of the childhood cancer due to shifting the calculated value of the components of the horizontal axis from the region of childhood cancer patients to the region of healthy children. On the other hand, the case when the calculated value of the components of the horizontal axis remained in the region of childhood cancer patients indicates that the therapeutic drug or the therapeutic method to be tested is not effective in the treatment of the childhood cancer.

As described above, therapeutic drugs or therapeutic methods for treating or preventing childhood cancer can be identified by the method for evaluating the efficacy of a treatment for a childhood cancer according to the present invention, and furthermore, the efficacy of the therapeutic drugs or the therapeutic method can be verified.

The present invention is described with reference to the following examples, however the examples are provided merely for the explanation of the present invention and are not intended to limit or restrict the scope of the present invention disclosed in the present application.

### EXAMPLES

### [Example 1] Comprehensive Analysis of Urinary Metabolites Related to Childhood Cancer

### (1-1) Comprehensive Analysis Scheme of Urinary Metabolites Related to Childhood Cancer

To identify the childhood cancer markers, a comprehensive analysis of urinary metabolites from 58 healthy children and 6 childhood cancer patients prior to treatment was performed at the Department of Pediatric Surgery, Nagoya University Graduate School of Medicine.

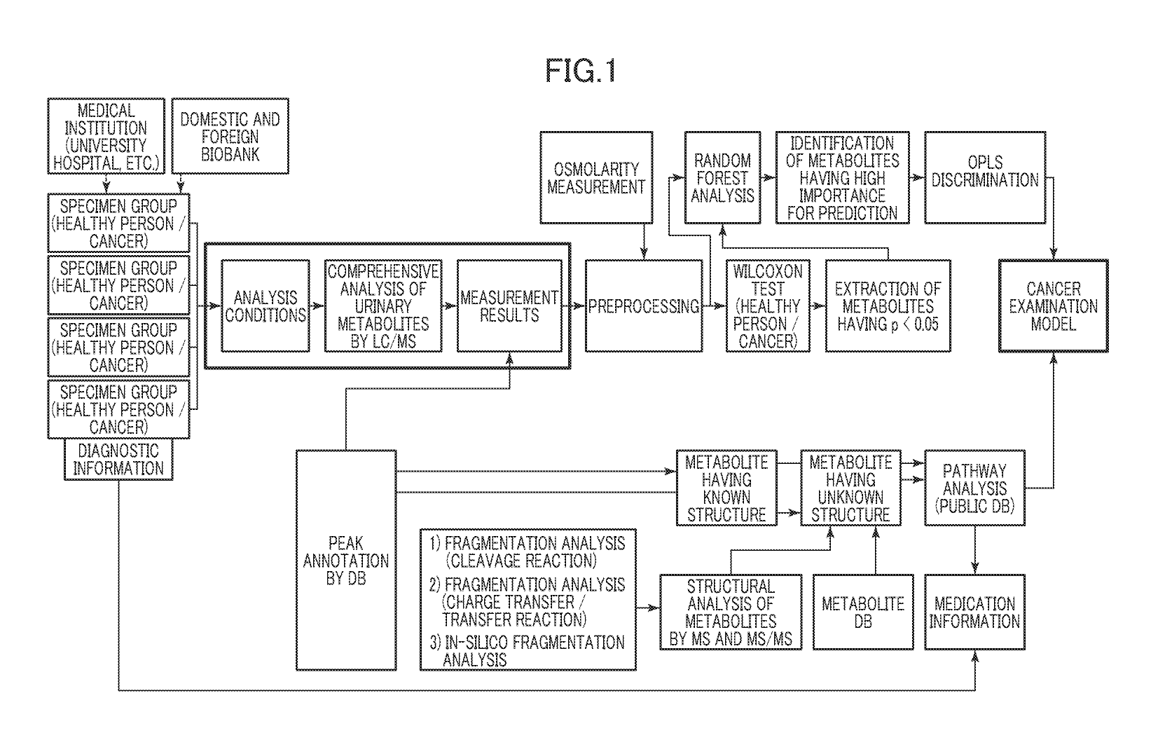

The search for tumor markers by the comprehensive analysis of the urinary metabolites was performed by the following (1) to (7). The flow is shown in FIG. 1. Specifically, urinary metabolites in the urine of children having a childhood cancer and of healthy children of the same age were comprehensively analyzed using a liquid chromatography mass spectrometer (LC/MS), the metabolites which significantly increased or decreased in the childhood cancer patients relative to the healthy children were identified, and the urinary tumor markers which can discriminate healthy children from childhood cancer patients were identified by multivariate analysis.

(1) A plurality of the specimens including urine specimen of healthy children and childhood cancer patients (before and after treatment) including additional clinical information were obtained.
(2) A comprehensive analysis was performed for the obtained urine specimen by a liquid chromatography mass spectrometer (LC/MS). Finally, as many metabolites as possible were measured from the data obtained by the LC/MS in order to discriminate the urine from the healthy children and the urine from the cancer patients. Therefore, a plurality of analyses were performed by combining a plurality of analysis modes (reverse-phase mode and hydrophilic interaction mode) in a liquid chromatography and a positive/negative electrospray ionization method in a mass spectrometer.
(3) Data preprocessing was performed for the obtained data, and in some cases, the metabolites were narrowed due to the p-value to the urinary metabolites having a significant difference in the cancer patients relative to the healthy children by the Wilcoxon rank sum test. The significance level at this time was set to 5%.
(4) While it is possible for a higher order test to narrow down the important tumor marker candidates, a quantitative evaluation is difficult, thus, a Random Forest (RF) method which is a machine learning method was used on the narrowed metabolites to quantitatively evaluate the importance of the metabolites. The higher the numerical value which is the importance, the more important the metabolite is, and thus, the metabolite is ranked in a higher order.
(5) Two types were extracted from among the metabolites extracted by Random Forest analysis, the precision variable R2Y and the prediction variable Q2 were calculated, and ordered in descending order of the value of the prediction variable to extract the higher order ranked metabolites.
(6) When it was determined from the results of the search of a public database, etc., that the structure of tumor marker candidate was a known substance, the metabolic pathway analysis was performed. If the metabolite is not derived from a drug ex vivo etc., it becomes a tumor marker candidate.
(7) Finally, an extracted tumor marker candidate group is used to calculate a discrimination formula for discriminating a healthy person from a cancer patient by the OPLS discrimination analysis method, and construct the cancer examination model. Among the urinary metabolites, the structure of the metabolites having unknown structures were estimated from the obtained positive and negative mass spectrum, and the positive and negative mass analysis/mass analysis spectrum (MS/MS spectrum) for the metabolites having an unknown structure which were not retrieved in a public database, etc. In-Silico fragmentation analysis was performed for the estimated structures, and the coincidence with the measured MS/MS spectrum was evaluated. When the coincidence was high, the estimated structure was deemed to be correct. When the structure could be estimated, the metabolic pathway analysis was performed, and the metabolite was deemed to be a tumor marker candidate.
(8) Taking the above results into consideration, a final quantitative assay for examination which includes the tumor marker was determined.

The following three points were noted for the statistical analysis in the results of the comprehensive analysis of the urinary metabolites by the above-mentioned LC/MS.

(i) Data Preprocessing

The results obtained by the comprehensive analysis with LC/MS are displayed with, for example, the information (metabolites having unknown structures and metabolites having known structures) relating to the metabolite, the information relating to the measurement conditions by the LC/MS, the diagnostic information in each specimen, and the measured ionic strength (area value of the observed peak) and the elution time in liquid chromatography in each metabolite.

When considering the measured ionic strength by the LC/MS, the dynamic range is large, thus, if calculated as is, the largest variable dominates over all calculations of the variance. It is necessary to eliminate the influence due to the dominant variable. First, the median value was set to 1, and the values of all the specimens were corrected. Further, the urine samples are expected to have different concentrations depending on the conditions in the body. In order to correct for this, a correction method by quantification of the creatinine in the urine or a correction method depending on the concentration of urinary solute measured by freezing point depression was carried out. In this case, the latter correction method was used. For the actual measurement, an automatic micro-osmometer Fiske 210 (sample amount: 20 uL, measurement time: 90 seconds, measurement range: 0 to 2000 mOsm/kgH₂O) by Advanced Instruments Inc. was used.

(ii) Examination and Machine Learning

In order to effectively specify the metabolites which significantly increased or decreased in the childhood cancer patients relative to the healthy children among the remarkable large number of metabolites detected, after narrowing the metabolites by the p-value (5%) to the urinary metabolites showing a significant difference in the cancer patients relative to the healthy children by the Wilcoxon rank sum test, a Random Forest (RF) method which is a machine learning method was used to quantitatively evaluate the importance of the metabolites. The higher the numerical value which is the importance, the more important the metabolite is, thus, it is ranked in a higher order. Two types were extracted from among the metabolites extracted by Random Forest analysis, the precision variable R2Y and the prediction variable Q2 were calculated, and ordered in descending order of the value of the prediction variable so as to extract the higher order ranked metabolites.

(iii) Multivariate Analysis

In the cancer examination scheme by the urinary metabolite, a multivariate analysis is used for visualizing the data or constructing a prediction model for regression and discrimination. There are several methods such as Principal Component Analysis (PCA) and Partial Least Squares (PLS) which are well-known as methods for multivariate analysis, and as methods used comparatively in metabolite analysis. PLS includes the PLS discrimination method, the OPLS discrimination analysis and the like as derived methods. PCA is a dimension reduction method based on variance maximization of the explanatory variable, whereas PLS is based on the maximization of the covariance of the objective variable and the explanatory variable. Therefore, the difference between PCA and PLS may be whether or not the group information is used in the calculation, and may be distinguished in that PCA is unsupervised and PLS is a supervised dimension reduction method.

In this analysis, all of the data is first overviewed in PCA, and OPLS-DA to which group information (healthy children and childhood cancer patients prior to treatment) is provided in addition to the metabolite data was used.

(1-2) Result of the Examination

A comprehensive analysis was performed for urine specimens from 58 healthy children and 6 childhood cancer patients (neuroblastoma) with a liquid chromatography mass spectrometer (LC/MS). As a result, the metabolites having a significant difference in the childhood cancer patients relative to the healthy children were narrowed from the detected metabolites (1331 types) to 272 types by the Wilcoxon rank sum test, and those were narrowed to the highest ranked 30 types by Random Forest. The results are shown in Table 2. In addition, the metabolic pathways verified by the public database search, the mass of the observed ions, and the analysis mode in this case are shown in Table 2. An entry in the table denoted with an X is a metabolite having an unknown structure. In the analysis mode, LC/MS Neg and LC/MS Pos respectively indicate the negative ion measurement and the positive ion measurement in the LC/MS.

TABLE 2
Analysis results by Random Forest

| | | Metabolic | Observed | Analysis |
|---|---|---|---|---|
| No. | Metabolite | pathway | mass | mode |
| 1 | N-acetilcysteine | Methionine | 162.02304 | LC/MS Neg |
| | | metabolism | | |
| 3 | 3-methoxytyramine sulfate | Tyrosine | 246.04416 | LC/MS Neg |
| | | metabolism | | |
| 4 | X-A | | — | — |
| 6 | vanillactate | Tyrosine | 211.06119 | LC/MS Neg |
| | | metabolism | | |
| 7 | xanthopterin | Pterin | 180.0516 | LC/MS Pos |
| | | metabolism | | |
| 8 | homovanillate (HVA) | Tyrosine | 181.05063 | LC/MS Neg |
| | | metabolism | | |
| 9 | X-B | | — | — |
| 10 | vanillylmandelate (VMA) | Tyrosine | 197.04555 | LC/MS Neg |
| | | metabolism | | |
| 11 | 3,4-dihydroxyphenylacetate | Tyrosine | 123.04515 | LC/MS Neg |
| | | metabolism | | |
| 12 | cystathionine | Methionine | 223.07471 | LC/MS Pos |
| | | metabolism | | |
| 13 | phenol glucuronide | Tyrosine | 269.06667 | LC/MS Neg |
| | | metabolism | | |
| 15 | cortisol 21-glucuronide | Corticosteroid | 537.23413 | LC/MS Neg |
| 17 | cortisol | Corticosteroid | 361.20204 | LC/MS Neg |
| 18 | S-adenosylhomocysteine | Methionine | 383.11431 | LC/MS Neg |
| | (SAH) | metabolism | | |
| 19 | 3,4-dihydroxyphenylacetate | Tyrosine | 246.99179 | LC/MS Neg |
| | sulfate | metabolism | | |
| 20 | 3-methoxytyrosine | Tyrosine | 212.09174 | LC/MS Pos |
| | | metabolism | | |
| 21 | X-C | | — | — |
| 22 | 3-methoxytyramine | Tyrosine | 168.10191 | LC/MS Pos |
| | | metabolism | | |
| 23 | X-D | | — | — |
| 24 | X-E | | — | — |
| 25 | 3-methoxy-4- | Tyrosine | 167.07028 | LC/MS Pos |
| | hydroxyphenylglycol | metabolism | | |
| 27 | dopamine | Tyrosine | 154.08626 | LC/MS Pos |
| | | metabolism | | |
| 28 | X-F | | — | — |
| 30 | X-G | | — | — |

As shown in Table 2, the 18 types of 3-methoxytyramine sulfate, cortisol, cortisol 21-glucuronide, N-acetylcysteine, 3-methoxytyrosine, phenol glucuronide, 3,4-dihydroxyphenylacetate, 3,4-dihydroxyphenylacetate sulfate, homovanillate, xanthopterin, vanillactate, 3-methoxytyramine, vanillylmandelate, S-adenosylhomocysteine, 3-methoxy-4-hydroxyphenylglycol, dopamine, and cystathionine with known structures, and the 7 types of X-A, X-B, X-C, X-D, X-E, X-F, and X-G with unknown structures were extracted as the urinary metabolites. Among the measured metabolites, those which are clearly considered to be exogenous metabolites such as drugs were excluded from the candidates.

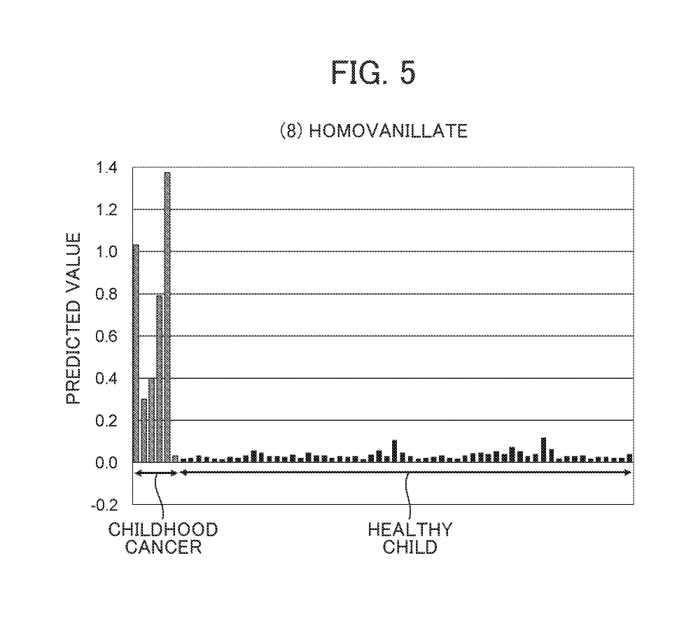

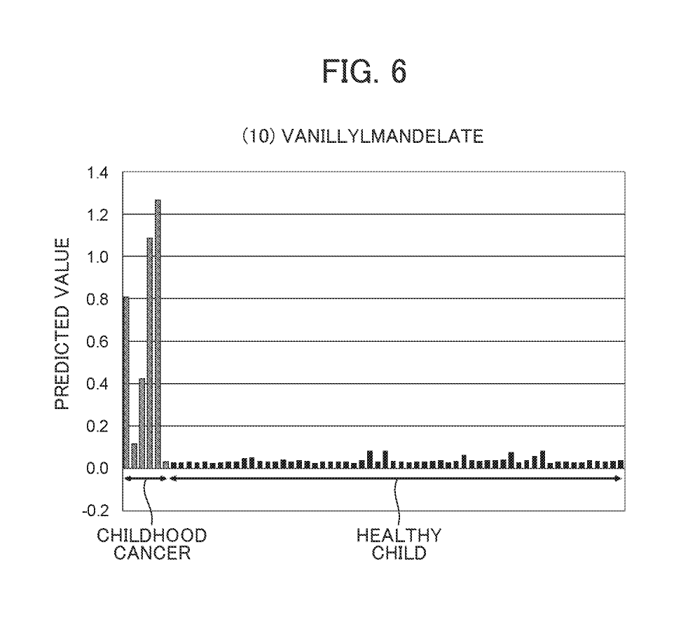

Neuroblastoma among the targeted childhood cancers this time is a solid tumor which can appear early in childhood, and neuroblastoma cells often produce a substance called catecholamine. As a result, the concentration of the substances vanillylmandelate (VMA) and homovanillate (HVA) in the urine is already known to become high (for example, patent documents described in the “Background Art”). It has been verified that these substances are involved in the tyrosine metabolic pathway, and both substances were detected in this comprehensive analysis. Homovanillate (HVA) and vanillylmandelate (VMA) are normally detected and quantified by a liquid chromatography mass spectrometer. Currently, the respective reference values have been determined. The results of detecting these metabolites by the LC/MS and performing OPLS discriminant analysis are respectively shown in FIGS. 5 and 6.

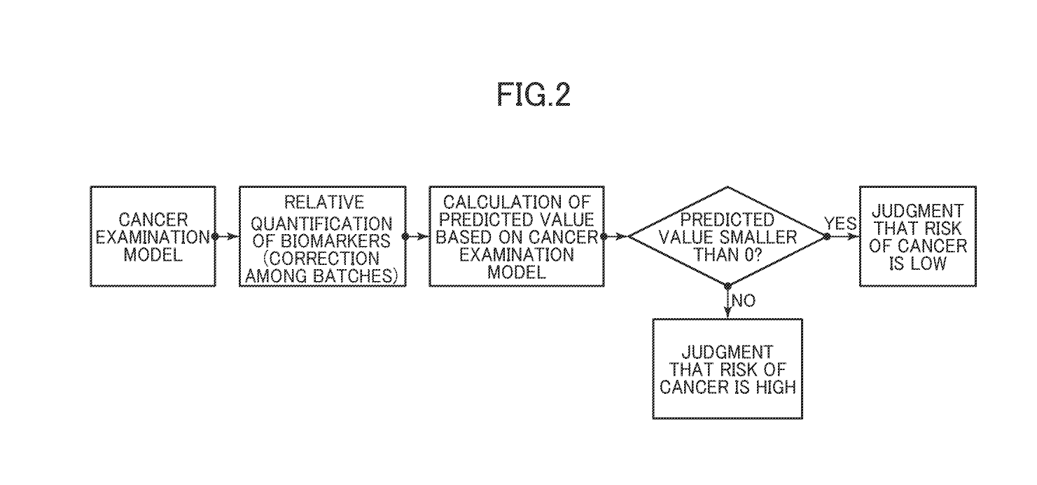

In addition, a cancer examination model was set for the case when a discrimination line for discriminating the healthy children from the childhood cancer patients was set to 0.05. When two or more metabolites are used, the discriminant analysis such as that used with the cancer examination model becomes possible. So, if the predicted value is greater than 0, the risk of cancer is judged to be high (for example, FIG. 2). However, only a single regression analysis is possible by the analysis of one metabolite. In this case, the results are as shown in FIG. 5, but if it is judged that the predicted value is larger than 0 and that the risk of cancer is high, it is judged that all of the healthy persons have a high risk of cancer. In this case, the risk was judged by setting a threshold suitable for discrimination, for example, to 0.05 in FIG. 5.

With respect to the discrimination of the healthy children and the childhood cancer patients, the results as in Tables 3 and 4 below were obtained. The cancer examination model in this case follows the following formula:

Cancer Examination Model (when the Number of Biomarkers is One)

Predicted value=α₁*(relative intensity of biomarker 1)+β₁

(wherein, α₁ and β₁ are constants)

TABLE 3
Prediction in the case of Vanillylmandelate (VMA)

| | Total | Predicted | |
|---|---|---|---|
| | number | number of | Predicted number |
| | of | healthy | of childhood |
| Classification | persons | children | cancer patients |
| Healthy child | 58 | 51 | 7 |
| Childhood cancer | 6 | 1 | 5 |

TABLE 4
Prediction in the case of Homovanillate (HVA)

| | Total | Predicted | Predicted |
|---|---|---|---|
| | number | number of | number of |
| | of | healthy | childhood |
| Classification | persons | children | cancer patients |
| Healthy child | 58 | 50 | 8 |
| Childhood cancer | 6 | 1 | 5 |

Furthermore, it is also possible to perform the multivariate analysis using a plurality of the urinary tumor markers. The cancer examination model formulas in this case are as follows:

Cancer Examination Model (when the Number of Biomarkers is Two)

Predicted value=α₂*(relative intensity of biomarker 1)+β₂*(relative intensity of biomarker 2)+γ₂

(wherein, α₂, β₂, and γ₂ are constants)

Cancer Examination Model (when the Number of Biomarkers is Three)

Predicted value=α₃*(relative intensity of biomarker 1)+β₃*(relative intensity of biomarker 2)+γ₃ (relative intensity of biomarker 3)+δ₃

(wherein, α₃, β₃, γ₃, and δ₃ are constants)

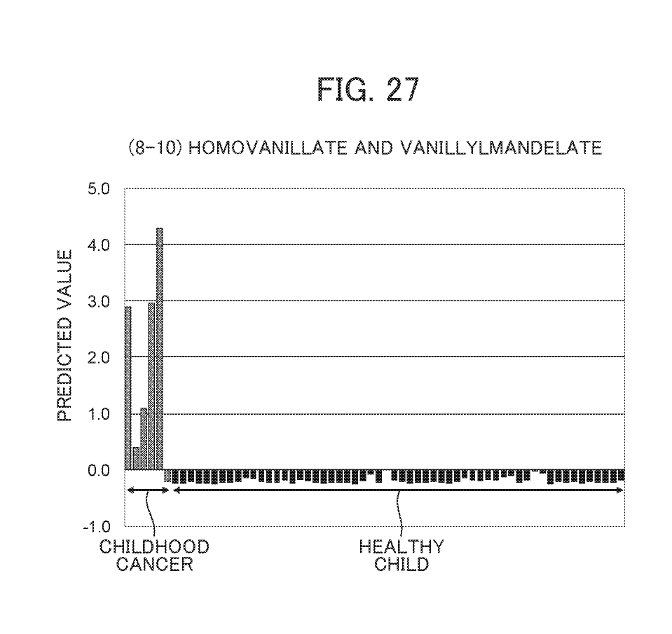

For example, when using two types of urinary tumor markers (vanillylmandelate (VMA) and homovanillate (HVA)) for the childhood cancer patients (6 persons with neuroblastoma) in the healthy children (58 persons), if calculated by the cancer examination model, the analysis results as shown in FIG. 27 could be obtained. Further, as shown in Table 5, the number of healthy children and childhood cancer patients can be obtained with a high accuracy. Specifically, it is understood that the error ratio in the prediction becomes small by increasing the number of powerful biomarkers from one to two. Therefore, it was confirmed that the predictability is high by combining these biomarkers or combining with other biomarkers to make a new cancer examination model even with existing biomarker VMA or HVA.

TABLE 5
Prediction by cancer examination model
(Combination of VMA and HVA)

| | Total | Predicted | Predicted number |
|---|---|---|---|
| | number of | number of | of childhood |
| Classification | persons | healthy children | cancer patients |
| Healthy child | 58 | 57 | 1 |
| Childhood cancer | 6 | 1 | 5 |

In the case of the OPLS discrimination analysis method, it is possible to evaluate with the cancer examination model as described above, but, when the number of biomarkers further increased, it is also possible to make the judgement using the following indications:

$$R 2 Y = 1 - \frac{\sum {( Yobs - Ycalc )}^{2}}{\sum {( Yobs - \overline{Y} )}^{2}}$$

$$Q 2 = 1 - \frac{\sum {( Yobs - Ypred )}^{2}}{\sum {( Yobs - \overline{Y} )}^{2}}$$

Herein, Yobs represents the measured value, Ycalc represents the value calculated by OPLS, Ypred represents the predicted value when the cross validation was performed, and Ŷ

represents the mean value. The cross validation illustrates a method for dividing data, analyzing a part of the data first, then, testing the analysis with the remaining portion of the data, and applying the results to verify and confirm the validity of the analysis itself. Accordingly, the closer the R2Y value is to 1, the higher the accuracy of the model, and the closer the Q2 value is to 1, the higher the predictability of the model.

Therefore, it is deemed that the suitability of the cancer examination model when the number of biomarkers increased utilizes a cancer examination model in which the predictability is high and is judged based on Q2. At this time, the case which was to be the criteria was considered as the case of the combination of VMA+HVA, and the R2Y value and the Q2 value were calculated at that time.

R2Y=0.621

Q2=0.326

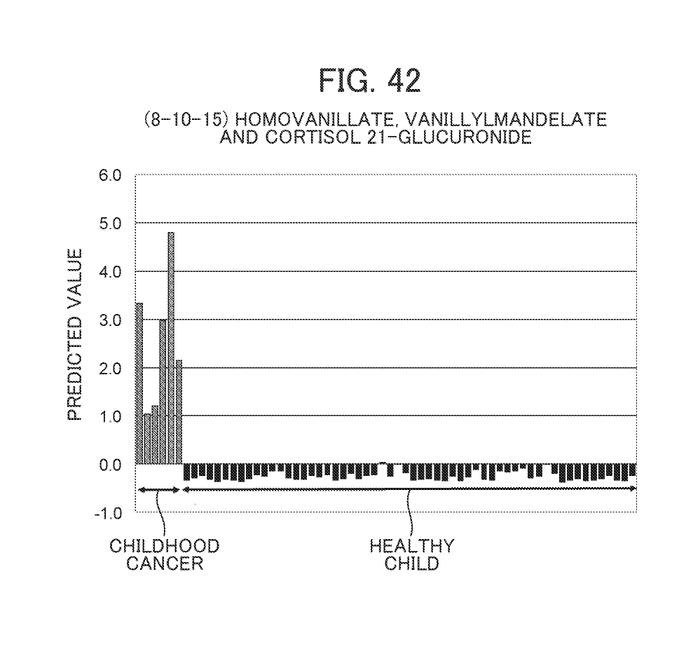

On the other hand, in addition to these two urinary tumor markers (tyrosine metabolism), if calculations are performed in the same manner using three types of biomarkers along with a metabolic substance (for example, cortisol 21-glucuronide) of another metabolic pathway where large changes were observed in the healthy children and the childhood cancer patients, the results as shown in FIG. 42 could be obtained. As shown in Table 6, the result further improved. Specifically, an improvement was observed by the evaluation of the healthy children (compared with FIG. 27).

TABLE 6
Analysis result by cancer examination model
(Combination of three types of markers)

| | | Predicted number | Predicted number |
|---|---|---|---|
| | Total number | of healthy | of childhood |
| Classification | of persons | children | cancer patients |
| Healthy child | 58 | 57 | 1 |
| Childhood cancer | 6 | 0 | 6 |

### [Example 2] Further Analysis of Urinary Tumor Markers Related to Childhood Cancer (1)

Only the urinary tumor markers having a known metabolite structure among the urinary tumor markers shown in Table 2 are shown in Table 7.

TABLE 7

| RF | | |
|---|---|---|
| number | Urinary tumor marker | Metabolic pathway |
| 1 | N-acetilcysteine | Methionine metabolism |
| 3 | 3-methoxytyramine sulfate | Tyrosine metabolite |
| 6 | vanillactate | |
| 7 | xanthopterin | Pterin metabolism |
| 8 | homovanillate (HVA) | Tyrosine metabolism |
| 10 | vanillylmandelate (VMA) | |
| 11 | 3,4-dihydroxyphenylacetate | Tyrosine metabolism |
| 12 | cystathionine | Methionine metabolism |
| 13 | phenol glucuronide | Tyrosine metabolism |
| 15 | cortisol 21-glucuronide | Corticosteroid |
| 17 | cortisol | Corticosteroid |
| 18 | S-adenosylhomocysteine (SAH) | Methionine metabolism |
| 19 | 3,4-dihydroxyphenylacetate sulfate | Tyrosine metabolism |
| 20 | 3-methoxytyrosine | Tyrosine metabolism |
| 22 | 3-methoxytyramine | |
| 25 | 3-methoxy-4-hydroxyphenylglycol | Tyrosine metabolism |
| 27 | dopamine | Tyrosine metabolism |

The analysis results by single regression when these urinary tumor markers were used alone in the cancer examination model are shown in FIGS. 3 to 19.

Further, two combinations among the urinary tumor markers shown in Table 7 were examined. There were 136 types of combinations overall, the R2Y and the Q2 of the combinations were obtained, and the combinations were arranged by numerical value in descending order.

TABLE 8
Promising combination of markers based on evaluation of R2Y and Q2

| Rank | Marker | Marker |
|---|---|---|
| 1 | 3-methoxytyramine sulfate | cortisol |
| 2 | 3-methoxytyramine sulfate | cortisol 21-glucuronide |
| 3 | 3-methoxytyramine sulfate | N-acetylcysteine |
| 4 | 3-methoxytyramine sulfate | 3-methoxytyrosine |
| 5 | 3-methoxytyramine sulfate | phenol glucuronide |
| 6 | 3-methoxytyramine sulfate | 3,4-dihydroxyphenylacetate |
| 7 | 3-methoxytyramine sulfate | 3,4-dihydroxyphenylacetate sulfate |
| 8 | 3-methoxytyramine sulfate | homovanillate (HVA) |
| 9 | 3-methoxytyramine sulfate | xanthopterin |
| 10 | 3-methoxytyramine sulfate | vanillactate |
| 11 | 3-methoxytyramine sulfate | 3-methoxytyramine |
| 12 | 3-methoxytyramine sulfate | vanillylmandelate (VMA) |
| 13 | 3-methoxytyramine sulfate | S-adenosylhomocysteine (SAH) |
| 14 | 3-methoxytyramine sulfate | 3-methoxy-4-hydroxyphenylglycol |
| 15 | 3-methoxytyramine sulfate | dopamine |
| 16 | homovanillate (HVA) | cortisol 21-glucuronide |
| 17 | 3,4-dihydroxyphenylacetate | cortisol 21-glucuronide |
| | sulfate | |
| 18 | 3-methoxytyramine | cortisol 21-glucuronide |
| 19 | vanillylmandelate (VMA) | 3-methoxytyrosine |
| 20 | 3-methoxytyrosine | 3-methoxy-4-hydroxyphenylglycol |
| 21 | 3-methoxytyramine | cortisol |
| 22 | vanillylmandelate (VMA) | cortisol 21-glucuronide |
| 23 | 3,4-dihydroxyphenylacetate | 3-methoxytyrosine |
| | sulfate | |
| 24 | 3,4-dihydroxyphenylacetate | cortisol |
| | sulfate | |
| 25 | homovanillate (HVA) | 3-methoxytyrosine |
| 26 | vanillylmandelate (VMA) | 3,4-dihydroxyphenylacetate sulfate |
| 27 | 3-methoxytyramine | S-adenosylhomocysteine (SAH) |
| 28 | homovanillate (HVA) | xanthopterin |
| 29 | xanthopterin | cortisol 21-glucuronide |
| 30 | 3,4-dihydroxyphenylacetate | 3-methoxy-4-hydroxyphenylglycol |
| | sulfate | |
| 31 | 3,4-dihydroxyphenylacetate | N-acetylcysteine |
| | sulfate | |
| 32 | 3,4-dihydroxyphenylacetate | 3-methoxytyrosine |
| 33 | homovanillate (HVA) | 3,4-dihydroxyphenylacetate sulfate |
| 34 | phenol glucuronide | 3,4-dihydroxyphenylacetate sulfate |
| 35 | vanillylmandelate (VMA) | phenol glucuronide |
| 36 | homovanillate (HVA) | phenol glucuronide |
| 37 | vanillylmandelate (VMA) | N-acetylcysteine |
| 38 | homovanillate (HVA) | N-acetylcysteine |
| 39 | 3,4-dihydroxyphenylacetate | xanthopterin |
| | sulfate | |
| 40 | 3,4-dihydroxyphenylacetate | S-adenosylhomocysteine (SAH) |
| | sulfate | |
| 41 | 3,4-dihydroxyphenylacetate | cortisol 21-glucuronide |
| 42 | dopamine | S-adenosylhomocysteine (SAH) |
| 43 | phenol glucuronide | 3-methoxytyramine |
| 44 | phenol glucuronide | 3-methoxy-4-hydroxyphenylglycol |
| 45 | 3-methoxy-4- | N-acetylcysteine |
| | hydroxyphenylglycol | |
| 46 | 3-methoxytyramine | N-acetylcysteine |
| 47 | 3-methoxy-4- | cortisol 21-glucuronide |
| | hydroxyphenylglycol | |
| 48 | 3-methoxytyrosine | xanthopterin |
| 49 | 3,4-dihydroxyphenylacetate | S-adenosylhomocysteine (SAH) |
| 50 | vanillylmandelate (VMA) | S-adenosylhomocysteine (SAH) |
| 51 | 3-methoxytyramine | dopamine |
| 52 | 3-methoxytyramine sulfate | cystathionine |
| 53 | vanillylmandelate (VMA) | xanthopterin |
| 54 | dopamine | cortisol 21-glucuronide |
| 55 | 3,4-dihydroxyphenylacetate | cortisol |
| 56 | vanillylmandelate (VMA) | 3,4-dihydroxyphenylacetate |
| 57 | phenol glucuronide | xanthopterin |
| 58 | 3-methoxy-4- | S-adenosylhomocysteine (SAH) |
| | hydroxyphenylglycol | |
| 59 | homovanillate (HVA) | 3,4-dihydroxyphenylacetate |
| 60 | 3,4-dihydroxyphenylacetate | phenol glucuronide |
| 61 | vanillylmandelate (VMA) | 3-methoxytyramine |
| 62 | 3-methoxytyramine | 3-methoxy-4-hydroxyphenylglycol |
| 63 | N-acetylcysteine | xanthopterin |
| 64 | 3,4-dihydroxyphenylacetate | N-acetylcysteine |
| 65 | 3-methoxy-4- | xanthopterin |
| | hydroxyphenylglycol | |
| 66 | 3-methoxytyrosine | 3-methoxytyramine |
| 67 | vanillylmandelate (VMA) | dopamine |
| 68 | dopamine | cortisol |
| 69 | 3,4-dihydroxyphenylacetate | 3,4-dihydroxyphenylacetate sulfate |
| 70 | 3,4-dihydroxyphenylacetate | 3-methoxytyramine |
| 71 | 3,4-dihydroxyphenylacetate | 3-methoxy-4-hydroxyphenylglycol |
| 72 | 3-methoxy-4- | dopamine |
| | hydroxyphenylglycol | |
| 73 | 3-methoxytyramine | xanthopterin |
| 74 | homovanillate (HVA) | 3-methoxytyramine |
| 75 | homovanillate (HVA) | S-adenosylhomocysteine (SAH) |
| 76 | 3-methoxytyrosine | S-adenosylhomocysteine (SAH) |
| 77 | homovanillate (HVA) | dopamine |
| 78 | 3,4-dihydroxyphenylacetate | 3-methoxytyramine |
| | sulfate | |
| 79 | S-adenosylhomocysteine | xanthopterin |
| | (SAH) | |
| 80 | phenol glucuronide | S-adenosylhomocysteine (SAH) |
| 81 | phenol glucuronide | dopamine |
| 82 | 3,4-dihydroxyphenylacetate | xanthopterin |
| 83 | vanillylmandelate (VMA) | 3-methoxy-4-hydroxyphenylglycol |
| 84 | homovanillate (HVA) | vanillylmandelate (VMA) |
| 85 | dopamine | N-acetylcysteine |
| 86 | 3,4-dihydroxyphenylacetate | cystathionine |
| | sulfate | |
| 87 | 3-methoxytyrosine | dopamine |
| 88 | N-acetylcysteine | S-adenosylhomocysteine (SAH) |
| 89 | vanillactate | 3-methoxytyramine |
| 90 | dopamine | xanthopterin |
| 91 | 3,4-dihydroxyphenylacetate | dopamine |
| | sulfate | |
| 92 | homovanillate (HVA) | cortisol |
| 93 | homovanillate (HVA) | 3-methoxy-4-hydroxyphenylglycol |
| 94 | phenol glucuronide | N-acetylcysteine |
| 95 | vanillactate | vanillylmandelate (VMA) |
| 96 | vanillactate | S-adenosylhomocysteine (SAH) |
| 97 | vanillactate | homovanillate (HVA) |
| 98 | vanillactate | 3-methoxy-4-hydroxyphenylglycol |
| 99 | 3,4-dihydroxyphenylacetate | dopamine |
| 100 | 3-methoxytyramine | cystathionine |
| 101 | dopamine | cystathionine |
| 102 | phenol glucuronide | cystathionine |
| 103 | N-acetylcysteine | cystathionine |
| 104 | vanillactate | xanthopterin |
| 105 | 3,4-dihydroxyphenylacetate | cystathionine |
| 106 | S-adenosylhomocysteine | cortisol 21-glucuronide |
| | (SAH) | |
| 107 | vanillactate | 3,4-dihydroxyphenylacetate sulfate |
| 108 | 3-methoxytyrosine | cortisol 21-glucuronide |
| 109 | vanillactate | cortisol 21-glucuronide |
| 110 | 3-methoxy-4- | cystathionine |
| | hydroxyphenylglycol | |
| 111 | vanillactate | cystathionine |
| 112 | vanillylmandelate (VMA) | cystathionine |
| 113 | 3-methoxytyrosine | cystathionine |
| 114 | phenol glucuronide | 3-methoxytyrosine |
| 115 | vanillactate | dopamine |
| 116 | xanthopterin | cortisol |
| 117 | vanillactate | phenol glucuronide |
| 118 | 3-methoxytyrosine | N-acetylcysteine |
| 119 | homovanillate (HVA) | cystathionine |
| 120 | vanillactate | cortisol |
| 121 | vanillactate | N-acetylcysteine |
| 122 | vanillactate | 3,4-dihydroxyphenylacetate |
| 123 | cystathionine | xanthopterin |
| 124 | cortisol 21-glucuronide | cortisol |
| 125 | phenol glucuronide | cortisol 21-glucuronide |
| 126 | vanillylmandelate (VMA) | cortisol |
| 127 | cystathionine | cortisol 21-glucuronide |
| 128 | N-acetylcysteine | cortisol 21-glucuronide |
| 129 | cystathionine | S-adenosylhomocysteine (SAH) |
| 130 | 3-methoxytyrosine | cortisol |
| 131 | 3-methoxy-4- | cortisol |
| | hydroxyphenylglycol | |
| 132 | vanillactate | 3-methoxytyrosine |
| 133 | phenol glucuronide | cortisol |
| 134 | N-acetylcysteine | cortisol |
| 135 | S-adenosylhomocysteine | (SAH) cortisol |
| 136 | cystathionine | cortisol |

By the aforementioned analysis, it is shown that the accuracy of the prediction increased due to the combination of markers. The combination that showed particularly high values in this analysis was the combination of 3-methoxytyramine sulfate and cortisol. Specifically, in this combination, R2Y=0.893 and Q2=0.851. The next highest was the combination of 3-methoxytyramine sulfate and cortisol 21-glucuronide. Specifically, in this combination, R2Y=0.888, Q2=0.832. From the above, the combination of 3-methoxytyramine sulfate and cortisol, and the combination of 3-methoxytyramine sulfate and cortisol 21-glucuronide were determined to be specifically powerful biomarkers.

During the analysis, we had focused on the metabolic pathways of the urinary metabolites. Specifically,

(i) the urinary tumor markers of the tyrosine metabolic pathway shown in 3, 6, 8, 10, 11, 13, 19, 20, 22, 25 and 27 in Table 7;

(ii) the urinary tumor markers of the methionine metabolic pathway (methionine, cysteine and SAM metabolism) shown in 1, 12 and 18 in Table 7;

(iii) the urinary tumor markers of the corticosteroid metabolic pathway shown in 15 and 17 in Table 7; and

(iv) the urinary tumor marker of the pterin metabolic pathway shown in 7 in Table 7

were focused on so as to analyze the results shown in FIGS. 3 to 19 and the predicted values calculated as in Table 8. From FIGS. 3 to 19, in the case of the metabolites involved in the tyrosine metabolic pathway, it tends to be observed that the value of the sixth childhood cancer patient becomes a negative value, or even if it is a positive value it is a small value. On the other hand, for the metabolites related to the methionine metabolic pathway and the metabolites related to the corticosteroid metabolic pathway, the value of the sixth childhood cancer patient is comparatively large. These features are summarized in Table 9.

TABLE 9

| | | Features of the |
|---|---|---|
| | Features of the distribution | distribution of the |
| Metabolic | of the predicted value of | predicted value of |
| pathway | childhood cancer patients | healthy children |
| Tyrosine | While the predicted value of | There are cases when |
| metabolite | first childhood cancer patient | the variation of the |
| | is relatively high, the | predicted value is |
| | predicted value of the sixth | high |
| | childhood cancer patient is | |
| | relatively low | |
| Methionine | While the predicted value of | The variation of the |
| metabolite | first childhood cancer patient | predicted value is |
| | is relatively low, the predicted | comparatively low |
| | value of the sixth childhood | |
| | cancer patient is not relatively | |
| | low | |
| Corticosteroid | While the predicted value of | The variation of the |
| metabolite | first childhood cancer patient | predicted value is |
| | is relatively low, the predicted | low |
| | value of the sixth childhood | |
| | cancer patient is relatively | |
| | high | |
| Pterin | While the predicted value of | There are cases when |
| metabolite | first childhood cancer patient | the variation of the |
| | is relatively high, the | predicted value is |
| | predicted value of the sixth | high |
| | childhood cancer patient is | |
| | relatively low | |

In light of the foregoing observations, a list of graphs showing the predicted values by single regression or OPLS discrimination is summarized in Table 10 for each metabolic system.

TABLE 10

| | | RF number (including plurality |
|---|---|---|
| No. | Metabolic pathway | of combinations) |
| 1 | Tyrosine | 3, 6, 8, 10, 11, 13, 19, 20, 22, 25, 27, |
| | | 3-6, 3-8, 3-10, 3-11, 3-13, 3-20, 3-27, |
| | | 8-10, 8-20, |
| | | 10-22, |
| | | 8-10-3 |
| 2 | Methionine | 1, 12, 18, |
| | | 1-12, 12-18 |
| 3 | Corticosteroid | 15, 17, |
| | | 15-17 |
| 4 | Pterin | 7 |
| 5 | Tyrosine + Methionine | 3-1, 3-12, 3-18, |
| | | 8-1, 8-12, |
| | | 8-10-12 |
| 6 | Tyrosine + Corticosteroid | 3-15, 3-17, |
| | | 8-10-15 |
| 7 | Tyrosine + Methionine + | 8-10-3-15, |
| | Corticosteroid | 8-10-3-12-1-15 |

In Table 10, in addition to tyrosine, methionine, corticosteroid, and pterin, tyrosine+methionine, tyrosine+corticosteroid, tyrosine+methionine+corticosteroid which are combinations of tyrosine, methionine, corticosteroid, and pterin are also described.

The combinations of the urinary tumor markers shown in Table 10 were evaluated by the cancer examination model in the same manner as mentioned above. The results are shown in FIGS. 20 to 44.

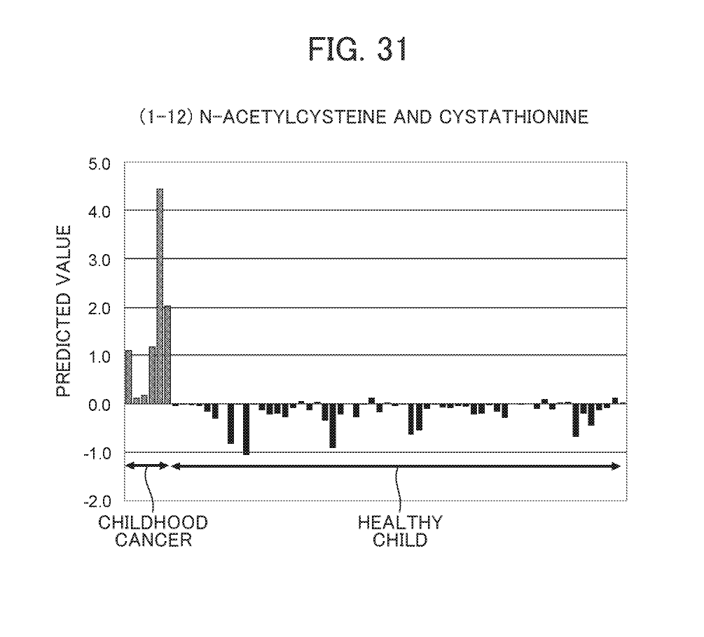

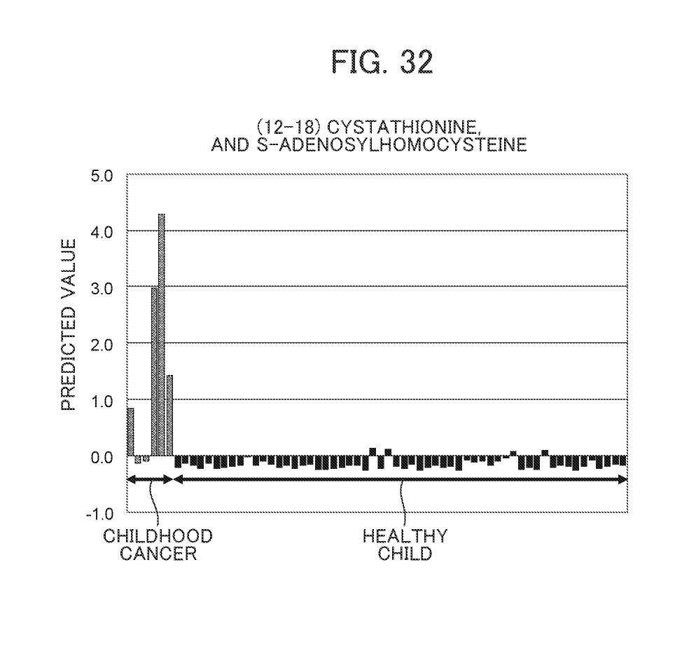

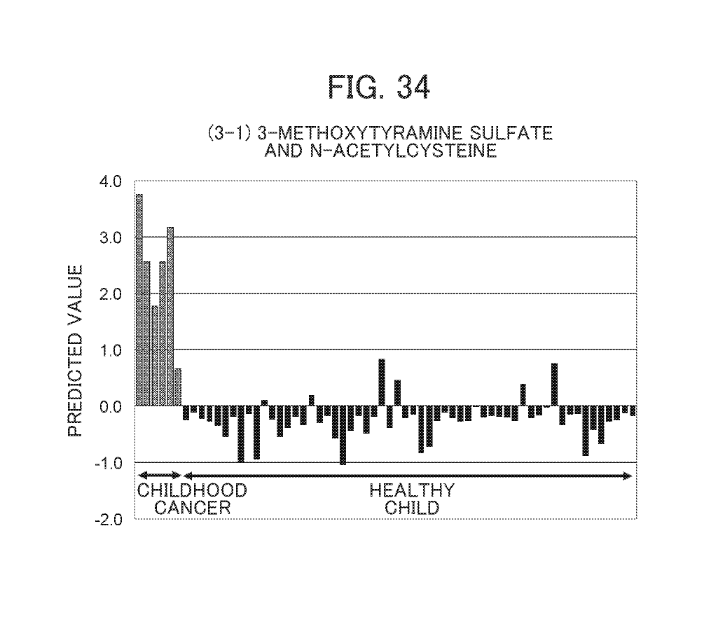

For example, when the urinary metabolite(s) of the tyrosine metabolic pathway and the urinary metabolite(s) of the methionine metabolic pathway were evaluated in combination (for example, FIGS. 34 to 39), it is understood that the appearance rate of false positives decreases, and a more accurate evaluation becomes possible compared to when only the urinary metabolites of the tyrosine metabolic pathway were evaluated in combination (for example, FIGS. 20 to 30) and when only the urinary metabolites of the methionine metabolic pathway were evaluated in combination (for example, FIGS. 31 and 32).

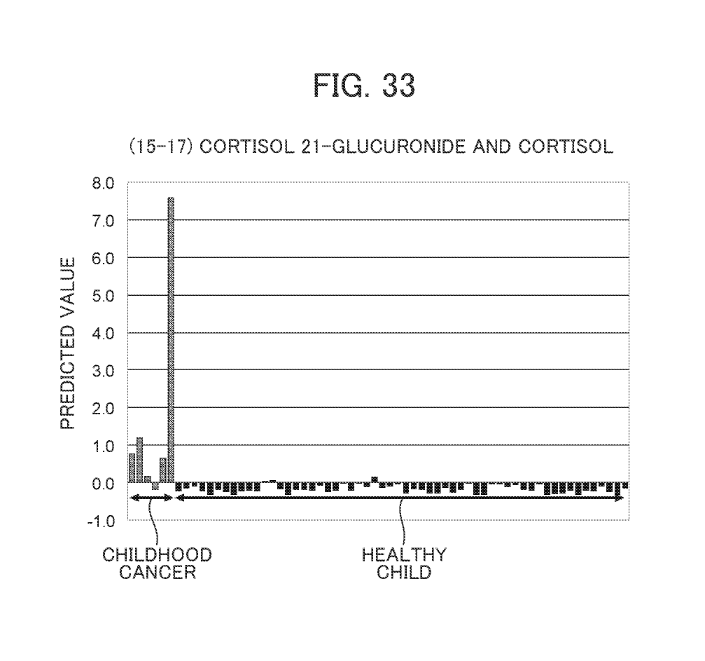

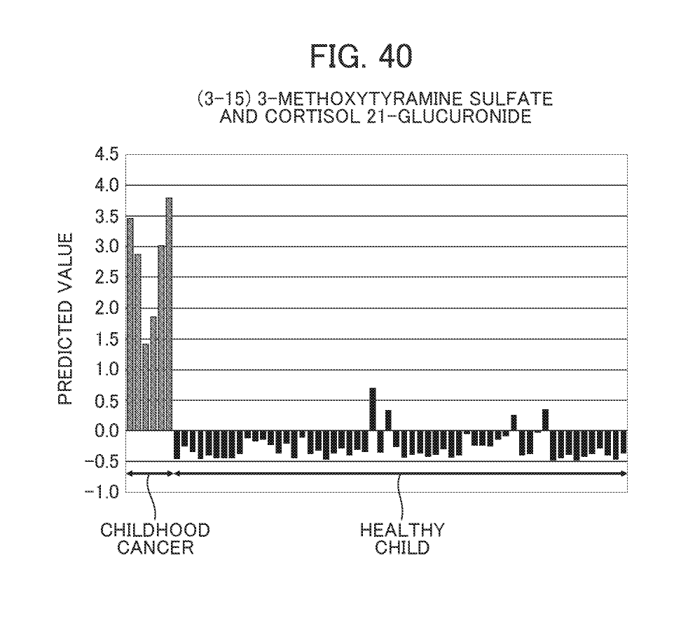

Similarly, when the urinary metabolite(s) of the tyrosine metabolic pathway and the urinary metabolite(s) of the corticosteroid metabolic pathway were evaluated in combination (for example, FIGS. 40 to 42), it is understood that the appearance rate of false positives decreases, and a more accurate evaluation becomes possible compared to when only the urinary metabolites of the tyrosine metabolic pathway were evaluated in the combination (for example, FIGS. 20 to 30) and when only the urinary metabolites of the corticosteroid metabolic pathway were evaluated in combination (for example, FIG. 33).

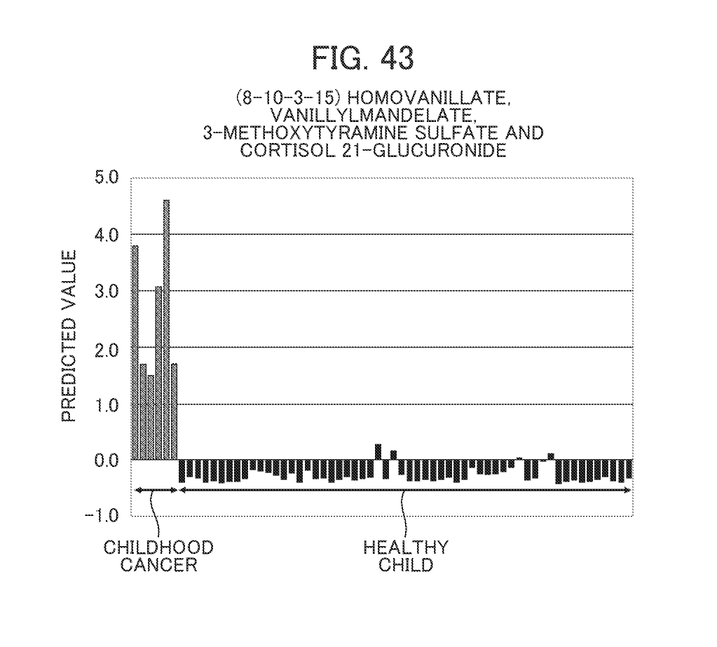

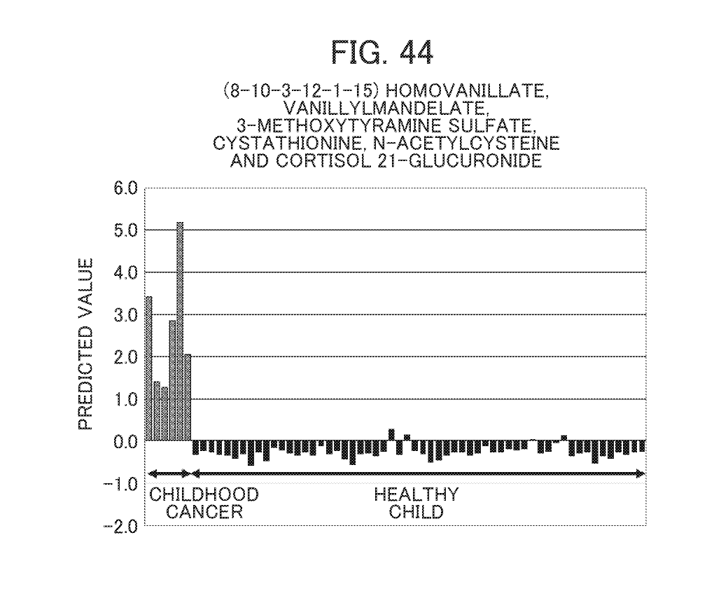

Further, when the urinary metabolite(s) of the tyrosine metabolic pathway, the urinary metabolite(s) of the methionine metabolic pathway and the urinary metabolite(s) of the corticosteroid metabolic pathway were evaluated in combination (for example, FIGS. 43 and 44), it is understood that the appearance rate of false positives decreases, and a more accurate evaluation becomes possible compared to when only the urinary metabolites of either metabolic pathway were combined and evaluated.

Therefore, when the urinary tumor markers were used in combination, it is understood that an evaluation having a higher accuracy becomes possible by combining urinary metabolites relating to different metabolic pathways.

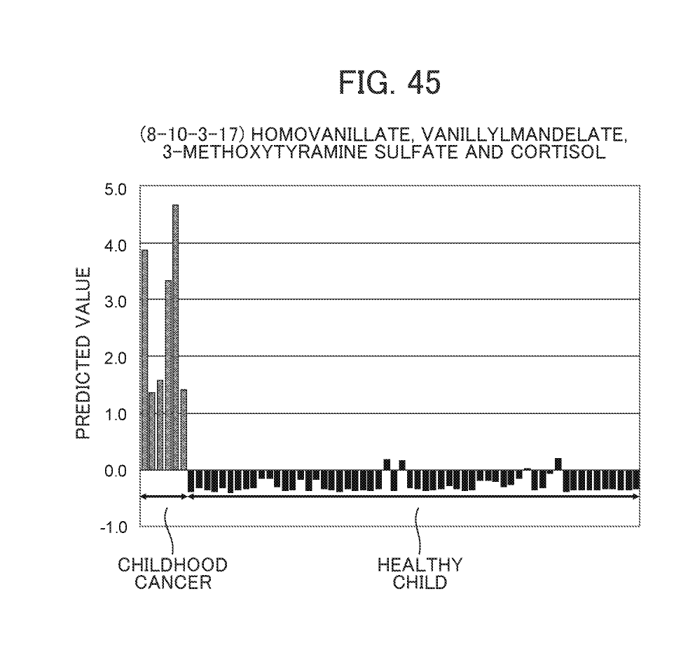

Furthermore, VMA and HVA are currently used clinically, thus, it is also useful to examine these combinations. For example, the case when the four biomarkers, VMA (tyrosine), HVA (tyrosine), 3-methoxytyramine sulfate (tyrosine) and cortisol (corticosteroid) were combined was also examined. As a result, high numbers were shown for R2Y=0.878 and Q2=0.786. In this case, the results by the OPLS discrimination method are shown in FIG. 45. It is understood that the ability to discriminate childhood cancers increased.

### [Example 3] Further Analysis of Urinary Tumor Markers Related to Childhood Cancer (2)

The urinary metabolites before and after treatment were analyzed for the 6 childhood cancer patients. Specifically, the 7 types of urinary tumor markers, homovanillate (VHA), vanillylmandelate (VMA), 3-methoxytyramine sulfate, cystathionine, N-acetylcysteine and cortisol 21-glucuronide were combined, and the predicted values were obtained in the same manner as Example 2.

The results are shown in FIG. 46. As is understood from the results, the two groups of the childhood cancer patients prior to treatment can be discriminated from the childhood cancer patients after treatment. Therefore, it is understood that the urinary tumor marker can be used to observe the course of the treatment of the childhood cancer, and can evaluate the treatment effect of the therapeutic drugs.

A previously conducted preliminary evaluation has already shown the possibility of creating a new research area of “childhood cancer diagnosis using a urine specimen” in the field of childhood cancer diagnosis which had no prior good diagnostic method.

Further, the collection methods for an examination using urine at a clinical site are remarkably simple relative to conventional examinations using blood, and the convenience for the medical staff also greatly improves.

All publications, patents, and patent applications cited herein are incorporated herein by reference in their entirety.

1. A method for evaluating a childhood cancer, comprising:
a step of measuring a urinary metabolite in a urine sample derived from a subject; and
a step of evaluating a childhood cancer in the subject on the basis of the result of the measurement,
wherein the urinary metabolite comprises at least one metabolite selected from the group consisting of:
(i) at least one metabolite selected from the group consisting of 3-methoxytyramine sulfate, vanillactate, homovanillate, vanillylmandelate, 3,4-dihydroxyphenylacetate, phenol glucuronide, 3,4-dihydroxyphenylacetate sulfate, 3-methoxytyrosine, 3-methoxytyramine, 3-methoxy-4-hydroxyphenylglycol, and dopamine;
(ii) at least one metabolite selected from the group consisting of N-acetylcysteine, cystathionine, and S-adenosylhomocysteine;
(iii) at least one metabolite selected from the group consisting of cortisol 21-glucuronide and cortisol; and
(iv) a metabolite which is xanthopterin,
provided that if the urinary metabolite to be evaluated is only one, it is not homovanillate or vanillylmandelate.

2. The method according to claim 1, wherein the urinary metabolite comprises:
a combination of at least one metabolite selected from the group of (i) and at least one metabolite selected from the group of (iii);
a combination of at least one metabolite selected from the group of (i) and at least one metabolite selected from the group of (ii);
a combination of at least two metabolites selected from the group of (i);
a combination of at least one metabolite selected from the group of (i) and at least one metabolite selected from the group of (iv);
a combination of at least one metabolite selected from the group of (iii) and at least one metabolite selected from the group of (iv);
a combination of at least one metabolite selected from the group of (ii) and at least one metabolite selected from the group of (iv);
a combination of at least two metabolites selected from the group of (ii);
a combination of at least one metabolite selected from the group of (ii) and at least one metabolite selected from the group of (iii);
a combination of at least two metabolites selected from the group of (iii);
a combination of at least one metabolite selected from the group of (i), at least one metabolite selected from the group of (ii) and at least one metabolite selected from the group of (iii);
a combination of at least one metabolite selected from the group of (i), at least one metabolite selected from the group of (ii) and at least one metabolite selected from the group of (iv);
a combination of at least one metabolite selected from the group of (i), at least one metabolite selected from the group of (iii) and at least one metabolite selected from the group of (iv);
a combination of at least one metabolite selected from the group of (ii), at least one metabolite selected from the group of (iii) and at least one metabolite selected from the group of (iv);
a combination of at least two metabolites selected from the group of (i) and at least one metabolite selected from the group of (iii);
a combination of at least two metabolites selected from the group of (i) and at least one metabolite selected from the group of (ii);
a combination of at least two metabolites selected from the group of (i) and at least one metabolite selected from the group of (iv); or
a combination of at least one metabolite selected from the group of (i), at least one metabolite selected from the group of (ii), at least one metabolite selected from the group of (iii) and at least one metabolite selected from the group of (iv).

3. The method according to claim 1, wherein the childhood cancer is neuroblastoma.

4. The method according to claim 1, wherein the subject is a 0 to 15-year-old child.

5. The method according to claim 1, wherein the evaluation of cancer comprises the detection of the childhood cancer in the subject, risk prediction of the childhood cancer in the subject, stage determination of the childhood cancer in the subject, prognosis determination of the childhood cancer in the subject, and/or monitoring of the effect of a treatment for the childhood cancer existing in the subject.

6. The method according to claim 1, wherein the evaluation step comprises a comparison with a reference value selected from the group consisting of: a measurement value of the urinary metabolite in samples from healthy children or low risk children, a measurement value of the urinary metabolite in samples of patients having a childhood cancer or having a childhood cancer at a known stage, a measurement value of the urinary metabolite in samples of patients having a childhood cancer showing a specific prognosis, a measurement value of the urinary metabolite in samples of patients who received a treatment for the childhood cancer, and a measurement value of the urinary metabolite in a sample of the subject which was obtained at another point in time.

7. The method according to claim 1, wherein the measurement of the urinary metabolite is performed by liquid chromatography mass spectrometry (LC/MS).

8. A device for evaluating a childhood cancer comprising:
a measurement unit which is configured to measure a urinary metabolite in a urine sample;
a comparison unit which is configured to compare a measurement value of the urinary metabolite measured by the measurement unit with a reference value or a previous measurement value; and
a determination unit which is configured to evaluate the childhood cancer from a comparison result obtained by the comparison unit,
wherein the urinary metabolite comprises at least one metabolite selected from the group consisting of:
(i) at least one metabolite selected from the group consisting of 3-methoxytyramine sulfate, vanillactate, homovanillate, vanillylmandelate, 3,4-dihydroxyphenylacetate, phenol glucuronide, 3,4-dihydroxyphenylacetate sulfate, 3-methoxytyrosine, 3-methoxytyramine, 3-methoxy-4-hydroxyphenylglycol, and dopamine;
(ii) at least one metabolite selected from the group consisting of N-acetylcysteine, cystathionine, and S-adenosylhomocysteine;
(iii) at least one metabolite selected from the group consisting of cortisol 21-glucuronide and cortisol; and
(iv) a metabolite which is xanthopterin,
provided that if the urinary metabolite to be evaluated is only one, it is not homovanillate or vanillylmandelate.

9. The device according to claim 8, wherein the urinary metabolites comprises:
a combination of at least one metabolite selected from the group of (i) and at least one metabolite selected from the group of (iii);
a combination of at least one metabolite selected from the group of (i) and at least one metabolite selected from the group of (ii);
a combination of at least two metabolites selected from the group of (i);
a combination of at least one metabolite selected from the group of (i) and at least one metabolite selected from the group of (iv);
a combination of at least one metabolite selected from the group of (iii) and at least one metabolite selected from the group of (iv);
a combination of at least one metabolite selected from the group of (ii) and at least one metabolite selected from the group of (iv);
a combination of at least two metabolites selected from the group of (ii);
a combination of at least one metabolite selected from the group of (ii) and at least one metabolite selected from the group of (iii);
a combination of at least two metabolites selected from the group of (iii);
a combination of at least one metabolite selected from the group of (i), at least one metabolite selected from the group of (ii) and at least one metabolite selected from the group of (iii);
a combination of at least one metabolite selected from the group of (i), at least one metabolite selected from the group of (ii) and at least one metabolite selected from the group of (iv);
a combination of at least one metabolite selected from the group of (i), at least one metabolite selected from the group of (iii) and at least one metabolite selected from the group of (iv);
a combination of at least one metabolite selected from the group of (ii), at least one metabolite selected from the group of (iii) and at least one metabolite selected from the group of (iv);
a combination of at least two metabolites selected from the group of (i) and at least one metabolite selected from the group of (iii);
a combination of at least two metabolites selected from the group of (i) and at least one metabolite selected from the group of (ii);
a combination of at least two metabolites selected from the group of (i) and at least one metabolite selected from the group of (iv); or
a combination of at least one metabolite selected from the group of (i), at least one metabolite selected from the group of (ii), at least one metabolite selected from the group of (iii) and at least one metabolite selected from the group of (iv).

10. The device according to claim 8, wherein the measurement unit comprises a liquid chromatography mass spectrometry (LC/MS) device.

11. A method for evaluating the efficacy of a treatment for a childhood cancer, comprising:
a step of measuring a urinary metabolite in a urine sample from an animal having a childhood cancer which was treated with a therapeutic drug or a therapeutic method to be tested; and
a step of evaluating the efficacy of the therapeutic drug or the therapeutic method to be tested for the childhood cancer on the basis of the result of the measurement,
wherein the urinary metabolite comprises at least one metabolite selected from the group consisting of:
(i) at least one metabolite selected from the group consisting of 3-methoxytyramine sulfate, vanillactate, homovanillate, vanillylmandelate, 3,4-dihydroxyphenylacetate, phenol glucuronide, 3,4-dihydroxyphenylacetate sulfate, 3-methoxytyrosine, 3-methoxytyramine, 3-methoxy-4-hydroxyphenylglycol, and dopamine;
(ii) at least one metabolite selected from the group consisting of N-acetylcysteine, cystathionine, and S-adenosylhomocysteine;
(iii) at least one metabolite selected from the group consisting of cortisol 21-glucuronide and cortisol; and
(iv) a metabolite which is xanthopterin,
provided that if the urinary metabolite to be evaluated is only one, it is not homovanillate or vanillylmandelate.

12. The method according to claim 11, further comprising a step of measuring the urinary metabolite in a urine sample from an animal having a childhood cancer prior to treatment with the therapeutic drug or the therapeutic method to be tested.

13. The method according to claim 11, wherein the animal having a childhood cancer is a human afflicted with a childhood cancer, or a childhood cancer model animal.
